(19) United States
(12) Patent Application Publication
(10) Pub. No.: US 2004/0072268 A1
(43) Pub. Date: Apr. 15, 2004

(54) Methods for regulating BRCA1-BRCA2-containing complex activity

(76) Inventor: Ramin Shiekhattar, Philadelphia, PA (US)

Correspondence Address: Licata & Tyrrell P.C., 66 E. Main Street, Marlton, NJ, 08053, US

(21) Appl. No.: 10/634,574

(22) Filed: Aug. 5, 2003

Related U.S. Application Data
(60) Provisional application No. 60/401,433, filed on Aug. 5, 2002.
(60) Provisional application No. 60/449,950, filed on Feb. 24, 2003.

(51) Int. Cl. G01N 33/574; A61K 48/00

(52) U.S. Cl. 435/7.23; 514/44

(57) ABSTRACT

The invention relates to methods of regulating the activity of at least one component of the BRCA1-BRCA2-containing complex (BRCC) by providing agents which specifically interact with BRCC components BRCC36 or BRE or nucleic acid sequences encoding BRCC36 or BRE. Methods of identifying such agents and using them to treat cancer are also provided. BRCC activities altered by such agents include ubiquitin E3 ligase activity, ubiquitin hydrolase activity, DNA repair activity, and transcriptional regulator activity.

BRCC36-specific antibodies and methods for diagnosing cancers associated with BRCC are also described.

### INTRODUCTION

This application claims the benefit of priority from U.S. provisional applications Serial No. 60/401,433, filed on Aug. 5, 2002 and Ser. No. 60/449,950, filed on Feb. 24, 2003.

This invention was made in the course of research sponsored by the National Institute of Health (NIH Grant No. CA 90758-02). The U.S. government may have certain rights in this invention.

### BACKGROUND OF THE INVENTION

Germline mutations in BRCA1 or BRCA2 genes predispose women to early onset, familial breast cancer (Hall, et al. (1990) Science 250:1684-1689; Narod, et al. (1991) Lancet 338:82-83; Miki, et al. (1994) Science 266:66-71; Wooster, et al. (1994) Science 265:2088-2090; Wooster, et al. (1995) Nature 378:789-792; Tavtigian, et al. (1996) Nat. Genet. 12:333-337). Moreover, deleterious alleles of BRCA1 and BRCA2 are responsible for almost all familial ovarian cancer, and deleterious alleles of BRCA2 are also involved in hereditary male breast cancer (Wooster, et al. (1995) Nature 378:789-792; Tavtigian, et al. (1996) Nat. Genet. 12:333-337; Easton, et al. (1993) Cancer Surv. 18:95-113; Miki, et al. (1994) Science 266:66-71).

Both BRCA1 and BRCA2 encode large proteins without extensive homology to other proteins (Miki, et al. (1994) Science 266:66-71; Tavtigian, et al. (1996) Nat. Genet. 12:333-337). The primary sequence of BRCA1 contains two motifs characteristic of transcription factors (Miki, et al. (1994) Science 266:66-71). These include a RING-finger motif and an acidic carboxyl terminus. Fusions of the carboxyl terminus to the DNA binding domain of GAL4 protein endows the chimeric protein with transcriptional stimulatory activity (Chapman and Verma (1996) Nature 382:678-679; Monteiro, et al. (1996) Proc. Natl. Acad. Sci. USA 26:13595-13599). BRCA1 may not only function as a coactivator of p53-mediated transcription (Ouchi, et al. (1998) Proc. Natl. Acad. Sci. USA 95:2302-2306; MacLachlan, et al. (2002) Mol. Cell. Biol. 22:4280-4292) but may also associate with RNA polymerase II (RNAPII) and the chromatin remodeling complex, SWI/SNF (Scully, et al. (1997) Proc. Natl. Acad. Sci. USA 94:5605-5610; Bochar, et al. (2000) Cell 102:257-265). Taken together, these observations indicate that BRCA1 may function as a transcriptional regulator.

Other reports indicate BRCA1 and BRCA2 may be involved in DNA repair. BRCA1 has been reported to interact with RAD51, BRCA2, and the RAD50 protein complex (Chen, et al. (1998) Mol. Cell 2:317-328; Zhong, et al. (1999) Science 285:747-750; Gowen, et al. (1998) Science 281:1009-1012; Scully, et al. (1997) Cell 88:265-275; Scully, et al. (1999) Mol. Cell 4:1093-1099; Sarkisian, et al. (2001) J. Biol. Chem. 276:37640-37648). BRCA1 mutant cells display sensitivity to DNA damaging agents and the BRCA1 protein has been reported to control homology-directed DNA repair (Moynahan, et al. (1999) Mol. Cell 4:511-518; Zhong, et al. (2002) J. Biol. Chem. May 30). Truncation of BRCA1 exon 11 results in defective G2-M cell cycle check point and an increased number of centrosomes (Xu, et al. (1999) Mol. Cell 3:389-395). Moreover, the BRCA2 protein interacts with RAD51 and plays a role in homology-directed repair (Wong, et al. (1997) J. Biol. Chem. 272:31941-31944; Chen, et al. (1998) Proc. Natl. Acad. Sci. USA 95:5287-5292; Mizuta, et al. (1997) Proc. Natl. Acad. Sci. USA 94:6927-6932; Marmorstein, et al. (1998) Proc. Natl. Acad. Sci. USA 95:13869-13874; Yu, et al. (2000) Genes Dev. 14:1400-1406; Moynahan, et al. (2001) Mol. Cell 7:263-272; Davies, et al. (2001) Mol. Cell 7:273-282). Furthermore, murine embryos with a targeted disruption of BRCA2 display sensitivity to ionizing radiation (Sharan, et al. (1997) Nature 386:804-810). Moreover, mouse embryo fibroblasts (MEFs) with a targeted disruption of BRCA2 exon 11 display increased sensitivity to ultraviolet light and methyl methanesulfonate (MMS) (Patel, et al. (1998) Mol. Cell 1:347-357).

BRCA1 interacts with the BRCA1-associated RING domain (BARD1) protein to form a heterodimeric complex (Wu, et al. (1996) Nat. Genet. 14:430-440; Brzovic, et al. (2001) Nat. Struct. Biol. 8:833-837). Remarkably, BARD1 association with BRCA1 potentiates the newly discovered ubiquitin E3 ligase activity of the BRCA1 protein (Hashizume, et al. (2001) J. Biol. Chem. 276:14537-14540). Detailed analysis of the BRCA1 ubiquitin E3 ligase activity has identified the RING domain of BRCA1 as the catalytic determinant for ubiquitination (Lorick, et al. (1999) Proc. Natl. Acad. Sci. USA 96:11364-11369; Hashizume, et al. (2001) J. Biol. Chem. 276:14537-14540; Ruffner, et al. (2001) Proc. Natl. Acad. Sci. USA 98:5134-5139). Furthermore, a recent report describes the ability of the BRCA1-BARD1 heterodimer to auto-ubiquitinate BRCA1 and BARD1 and trans-ubiquitinate the histone H2A(X) (Chen, et al. (2002) J. Biol. Chem. 277:22085-22092).

To date, there is no effective means of regulating the activities associated with BRCA1 or BRCA2. The present invention meets this need.

### SUMMARY OF THE INVENTION

The present invention relates to methods for regulating the activity of at least one component of the BRCA1-BRCA2-containing complex (BRCC). The method involves contacting BRCC or a cell containing BRCC with an agent that interacts with a nucleic acid sequence encoding BRCC36 or BRE, or a product thereof, thereby altering an activity of BRCC.

Further provided are methods for identifying agents which modulate BRCC-associated activities. Agents are selected for the ability to alter the ubiquitin E3 ligase activity or ubiquitin hydrolase activity of BRCC as determined by changes in the level of ubiquitination of a select protein, the DNA repair activity of BRCC as determined by changes in cell survival rates upon exposure to ionizing radiation or changes in homology-directed DNA repair, or the transcriptional regulator activity of BRCC as measured by changes in the expression of genes containing p53 response elements. In addition, a method of using such agents to treat cancer is described.

Also provided are antibodies specific for BRCC36 and a method of diagnosing a cancer or risk of developing a cancer associated with BRCC.

These and other aspects of the present invention are set forth in more detail in the following description of the invention.

### BRIEF DESCRIPTION OF THE DRAWINGS

FIG. 1 shows that BRCC36 inhibits BRCA1 transcriptional regulatory activity via p53. Luciferase assays were conducted with luciferase as the reporter operably linked to an MDM2 promoter.

### DETAILED DESCRIPTION OF THE INVENTION

A complex of proteins associated with BRCA2 and BRC1 have now been identified. This complex termed BRCC for BRCA1-BRCA2-containing complex contains BRCA2, BRCA1 and RAD51 as well as BRCC300, BRCC140, BRCC130, BRCC120, BRCC80, BRCC45 and BRCC36.

The present invention provides proteins encoded by the genes c6.1A (accession number S68015), referred to herein as BRCC36, and BRE (accession number NM_{—}004899). Using stable cell lines expressing epitope tagged BARD1, a multiprotein complex containing BRCA2 (SEQ ID NO:1), BRCA1 (SEQ ID NO:2), and RAD51 (SEQ ID NO:3) as well as BRCC300 (SEQ ID NO:4), BRCC140 (SEQ ID NO:5), BRCC130 (SEQ ID NO:6), BRCC120 (SEQ ID NO:7), BRCC80 (SEQ ID NO:8), BRE (SEQ ID NO:9) and BRCC36 (SEQ ID NO:10) was isolated. The complex was found to have an associated ubiquitin E3 ligase activity which was regulated by the negative regulator, BRCC36, as well as DNA repair and transcriptional regulator activities.

BRCC36 and BRE were initially isolated by identifying components that complex with BARD1. To isolate BARD1-containing complex(es), H1299- and 293-derived cell lines were developed which express FLAG®-tagged BARD1. Nuclear extract from native H1299 cells was used as the control for anti-FLAG® affinity purification. Analysis of the FLAG®-BARD1 eluate by sodium dodecyl sulfate-polyacrylamide gel electrophoresis (SDS-PAGE) and silver staining revealed the specific association of BARD1 with polypeptides of 350, 300, 210, 140, 130, 120, 80, 45, 40 and 36 kDa. A combination of mass spectrometric sequencing and western blot analysis identified the 350, 210, and 40 kDa bands as BRCA2 (SEQ ID NO:1; accession number P51587), BRCA1 (SEQ ID NO:2; accession number P38398), and RAD51 (SEQ ID NO:3; accession number Q06609), respectively. This BRCA1-BRCA2-containing complex will be referred to herein as BRCC. Analysis of a number of preparations indicated that RAD51 was a substoichiometric component of BRCC. The 300, 140, 130, 120, 80, 45, and 36 kDa polypeptides corresponded to predominantly unknown proteins designated DKFZp434D193.1 (SEQ ID NO:4), FLJ21816 (SEQ ID NO:5; accession number NP_{—}078951), ubiquitin hydrolase (SEQ ID NO:6; protein FLJ23277 with accession number NP_{—}115612), BRCA1 Δ11 (SEQ ID NO:7; accession number NP_{—}009234), RAP80 (SEQ ID NO:8; accession number AAK61871); BRE (SEQ ID NO:9; Li, et al. (1995) Biochem. Biophys. Res. Commun. 206:764-74; accession number NP_{—}004890) and C6.1A (SEQ ID NO:10; Fisch, et al. (1993) Oncogene 8:3271-6; accession number P46736), respectively. Accordingly, these proteins are referred to herein as BRCC300, BRCC140, BRCC130, BRCA Δ11, BRCC80, BRE, and BRCC36, respectively.

To establish that BRCC represented a single complex and was not specific to H1299 cells, BRCC was isolated from a 293-derived cell line expressing FLAG®-BARD1. BRCC was fractionated from cell extract by anion exchange chromatography using high stringency conditions (500 mM KCl). It was found that BRCA2, BRCA1, BARD1, BRE, BRCC36 and RAD51 coelute as a large multiprotein complex, peaking in fractions 18 through 20. A small fraction of RAD51 dissociates from the complex and elutes earlier, starting at fraction 10, consistent with a modular nature for the RAD51 association with BRCC. A fraction of BRCA1 and and BARD1 eluted at a smaller molecular mass (peak fraction 16) indicating that a fraction of BRCA1/BARD1 can be resolved from the larger complex. Further, immunoprecipitation experiments using anti-BARD1 and anti-BRCA1 antibodies demonstrated the association of BRCA1, BRCA2, and BARD1 from nuclear extract of native 293 cells. Taken together, these results demonstrated the stable association of BRCA1, BRCA2, BARD1, and RAD51 in a multiprotein complex.

A 293-derived cell line stably expressing FLAG®-BRCC36 was created to demonstrate the association of the novel components of the complex with BRCA1 and BARD1. BRCC36 displayed sequence homology with the Poh1/Pad1 subunit of the 26S proteasome and subunit 5 (Jab1) of the COP9 signalosome. The homology is found in the Jab1/MPN or the JAMM domain (Verma, et al. (2002) Science 298:611-5; Cope, et al. (2002) Science 298:608-11; Yao and Cohen (2002) Nature 419:403-7). Despite its homology to Poh1 and Jab1, BRCC36 represents a distinct branch in the evolutionary tree. The BRCC36 gene was located at the Xq28 locus, a chromosomal break-point in patients with pro-lymphocytic T-cell leukaemia (Fisch, et al. (1993) supra). In two cases of pro-lymphocytic T-cell leukaemia a chromosomal break occurred in two different introns of BRCC36 (Fisch, et al. (1993) supra).

Isolation of FLAG®-BRCC36 by FLAG®-affinity purification followed by mass spectrometric sequencing and western blot analysis demonstrated the specific and stable association of BRCC36 with the other components of BRCC including BRCA2, BRCA1, BARD1 and BRE. Further, endogenous BRCC36 was detected in FLAG®-BRCC36 affinity eluate, indicating the presence of more than one BRCC36 protein per BRCC. These results demonstrate that BRCC36 and BRE are bonafide components of BRCC.

The region encoded by the exon 11 of BRCA1 is a frequent target of cancer-causing mutations. To assess the integrity of the polypeptide composition of BRCC following such cancer-causing truncations of BRCA1, a 293-derived cell line was constructed which stably expressed a truncated BRCA1 (1-509) leaving the nuclear localization signal and the Ring domain intact. To examine whether a truncation of the BARD1 protein would have a similar effect to that of BRCA1, cell lines stably expressing a truncated form of BARD1 (1-398) containing the Ring domain and nuclear localization signal were constructed. Both truncated protein complexes were purified and the resulting polypeptides were analyzed for protein composition. While truncation of BARD1 did not affect the association of any of the components of the complex, the BRCA1 truncation completely abrogated the association of BRCC36 and BRE and reduced the association of both BRCA2 and RAD51. These results demonstrate that cancer causing truncations of the BRCA1 C-terminal domain destabilize the protein composition of BRCC. However, similar C-terminal truncations of BARD1 are not deleterious to complex formation.

The interaction of the BRCC subunits were further characterized by determining whether BRCC36 directly associates with BRCA1. Six fragments spanning BRCA1 were produced in bacteria and were tested for their association with recombinant BRCC36 (Table 1).

TABLE 1

| | BRCA1 amino acid | Association |
|---|---|---|
| Fragment | residues | with BRCC36 |
| F1 | 1-324 | − |
| F2 | 260-553 | − |
| F3 | 502-802 | + |
| F4 | 758-1064 | + |
| F5 | 1005-1313 | − |
| F6 | 1314-1863 | − |

To determine whether the BRCA1 interaction was specific to BRCC36 and not other JAMM domain-containing proteins, the Jab1/CSN5 subunit of signalosome was also produced in recombinant form for use in the protein-protein interaction assay. While BRCC36 specifically associated with fragments 3 and 4 encoded by exon 11 of BRCA1, Jab1/CSN5 did not associate with any fragments of BRCA1 protein. These data are consistent with the results showing the loss of BRCC36 following truncations of BRCA1 exon 11.

The mRNA expression levels of BRCC36 was analyzed in various breast tumor cell lines (SK-BR-3, T47D, MCF-7 and MDA-MB-468) and breast epithelial cell lines (MCF-12F, MCF-12A and MCF-10F), breast mammary organoids (OG1 and OG2), primary breast epithelial cells, and breast tumors using real-time PCR. In general, all tissues had an increase in BRCC36 expression. Quantitative real-time PCR was employed to further analyze normal mammary ductal epithelial cells and malignant epithelial cells captured by micro-dissection. It was found that 70-80% of the sporadic mammary tumors had a 50-70% increase in BRCC36 expression levels as compared to normal tissue indicating that an increase in BRCC36 expression is indicative of breast cancer or indicative of an increased risk of developing breast cancer.

A BRCA1-BARD1 heterodimer has been described having a ubiquitin E3 ligase activity (Lorick, et al. (1999) Proc. Natl. Acad. Sci. USA 96:11364-11369; Ruffner, et al. (2001) Proc. Natl. Acad. Sci. USA 98:5134-5139; Hashizume, et al. (2001) J. Biol. Chem. 276:14537-14540). It was determined whether BRCC displayed E3 ubiquitin ligase activity and whether its enzymatic activity was similar to that of recombinant BRCA1-BARD1. Recombinant G-BRCA1/F-BARD1 was generated by co-expressing GST-tagged BRCA1(1-639) and FLAG®-tagged BARD1 in bacteria (Chen, et al. (2002) J. Biol. Chem. 277:22085-22092). Both recombinant G-BRCA1/F-BARD1 and BRCC demonstrated Ubc5-dependent ubiquitin E3 ligase activity. Of those tested (Ubc3, Ubc5A, Ubc5B, Ubc5c, UbcH7, UbcH2), Ubc5c was the most active with either recombinant G-BRCA1/F-BARD1 or the BRCC complex as the E3 enzyme. These results demonstrate that BRCC is a ubiquitin E3 ligase complex.

A close comparison of the BRCC ubiquitin ligase activity to that of recombinant G-BRCA1/F-BARD1 indicated a higher activity for recombinant G-BRCA1/F-BARD1. To further analyze this difference, the ubiquitin ligase activity of each enzyme was normalized for equal concentrations of BRCA1 and BARD1. At lower enzyme concentrations, both BRCC and G-BRCA1/F-BARD1 displayed similar E3 ubiquitin ligase activity. However, increasing concentrations of G-BRCA1/F-BARD1 resulted in higher enzyme activity while the BRCC activity reached a plateau early in the titration of the enzyme. These results indicated that the activity of BRCC may be regulated by a modulatory subunit inhibiting the enzyme at a higher enzyme/modulator concentration.

As the Jab1 subunit of the COP9 complex modulates the degradation of p27 protein (Tomoda, et al. (1999) Nature 398:160-165) and sequence similarity exits between BRCC36 and Jab1, it was determined whether BRCC36 modulated the ubiquitin ligase activity of BRCC. Accordingly, BRCC36 was expressed and purified from bacteria and analyzed for its modulation of BRCC ubiquitin ligase activity. Increasing concentrations of BRCC36 inhibited the ubiquitin ligase activity of G-BRCA1/F-BARD1 in a dose-dependent manner. Although BRCC contains BRCC36, increasing concentrations of BRCC36 also inhibited the ubiquitin ligase activity of BRCC. The inhibition of E3 ubiquitin ligase activity by BRCC36 was specific since it did not affect the ubiquitin ligase activity of another ring-containing ubiquitin ligase, the mitotic check point protein, Chfr. To rule out the possibility that the inhibition of ubiquitination is a result of a deubiquitinating activity present in the BRCC36 preparation, BRCC36 was added to the ubiquitination reaction following the formation of the ubiquitin conjugates. Although BRCC36 inhibited the ubiquitination reaction when added at the beginning of the reaction, addition of BRCC36 two hours into the ubiquitination reaction did not effect the ubiquitination reaction.

A functional and physical association of BRCA1 and BRCA2 with the p53 protein has been described (Marmorstein, et al. (1998) Proc. Natl. Acad. Sci. USA 95:13869-13874; Ouchi, et al. (1998) Proc. Natl. Acad. Sci. USA 95:2302-2306; Bochar, et al. (2000) Cell 102:257-265). Therefore, it was determined whether p53 can serve as the substrate for ubiquitination by BRCC in vitro and whether this activity could be inhibited by BRCC36. Both G-BRCA1/F-BARD1 and BRCC specifically ubiquitinated p53. Moreover, BRCC36 inhibited the ubiquitination of p53 by either G-BRCA1/F-BARD1 or BRCC. Kinetic analysis indicated that the BRCC36-mediated inhibition could not be overcome by increasing concentrations of p53, indicating a non-competitive nature of inhibition by BRCC36. Accordingly, BRCC36 functions as a specific modulator of BRCC ubiquitin ligase activity.

The inhibition of ubiquitin ligase activity of individual BRCA1 or BARD1 subunits was examined to determine whether BRCC36 directly interacts with BRCA1 or BARD1. Each protein was independently purified from bacteria as a fusion with GST and was used in a ubiquitin ligase reaction. Although recombinant G-BARD1 did not display ubiquitin ligase activity, similar concentration of G-BRCA1 exhibited modest E3 ligase activity. Increasing concentrations of G-BARD1 alone was also devoid of any activity. However, addition of G-BARD1 to G-BRCA1 potentiated the ubiquitin ligase activity of BRCA1 to levels obtained using G-BRCA1/F-BARD1 purified after coexpression of the two proteins. Furthermore, addition of increasing concentrations of BRCC36 inhibited the ubiquitin ligase activity of G-BRCA1, indicating that the two proteins may directly interact. Protein-protein interaction experiments demonstrated that BRCC36 directly and specifically interacted with both recombinant BRCA1 and recombinant BRCA1-BARD1 heterodimer. Together, these data indicate a direct interaction of BRCA1 and BRCC36 resulting in the inhibition of the BRCA1 ubiquitin E3 ligase activity.

BRCA1 mutant cells display sensitivity to DNA damaging agents, and the BRCA1 protein has been reported to control homology-directed DNA repair (Moynahan, et al. (1999) Mol. Cell 4:511-8; Zhong, et al. (2002) J. Biol. Chem. 277:28641-7). Truncation of BRCA1 exon 11 has also been shown to result in defective G2/M cell cycle checkpoint and an increased number of centrosomes (Xu, et al. (1999) Mol. Cell 3:389-95). Moreover, the BRCA2 protein interacts with RAD51 and plays a role in homology-directed repair (Sharan, et al. (1997) Nature 386:804-10; Wong, et al. (1997) J. Biol. Chem. 272:31941-4; Chen, et al. (1998) Proc. Natl. Acad. Sci. USA 95:5287-92; Mizuta, et al. (1997) Proc. Natl. Acad. Sci. USA 94:6927-32; Patel, et al. (1998) Mol. Cell 1:347-57; Marmorstein, et al. (1998) Proc. Natl. Acad. Sci. USA 95:13869-74; Yu, et al. (2000) Genes Dev. 14:1400-6; Moynahan, et al. (2001) Mol. Cell 7:263-72; Davies,. et al. (2001) Mol. Cell 7:273-82). Accordingly, the responsiveness of cell lines overexpressing BARD1 or BRCC36 to ionizing radiation was examined. FLAG®-BARD1 or FLAG®-BRCC36 cell lines were exposed to ionizing radiation (4 and 8 grays) and cellular survival was determined. Cells overexpressing BARD1 or BRCC36 exhibited opposite responsiveness following ionizing radiation. While FLAG®-BARD1 cells displayed an enhancement in cellular survival, FLAG®-BRCC36 cells showed higher sensitivity to ionizing radiation than parental 293 cells.

Further, to determine whether loss of BRCC36 and BRE resulted in DNA repair defects, HeLa cells were treated by siRNA against BRCA1, BRCC36, BRE or control siRNA and their responsiveness to ionizing radiation was measured. The experiments were performed in triplicate comparing the cells treated with siRNA against BRCC36 or BRE, to those treated with siRNA against BRCA1 and control siRNA. Treatment of cells with siRNA against BRCA1, BRCC36 and BRE resulted in decreased cell number even in the absence of ionizing radiation. However, BRCA1-, BRCC36- and BRE-depleted cells displayed a potent increased sensitivity to ionizing radiation at all doses examined.

Moreover, it was ascertained whether depletion of BRCC36 and BRE would result in disruption of the G2/M checkpoint arrest. Consistent with a defect in G2/M checkpoint, analysis of mitotic cells following 2 and 4Gy of ionizing radiation indicated that approximately three-fold more BRCC36- and BRCA1-depleted cells entered into mitosis. Depletion of BRE resulted in a more moderate defect in G2/M checkpoint arrest. Taken together these results demonstrate that BRCA1, BRCC36 and BRE are not only components of a multiprotein complex but also participate in a similar pathway of cellular responsiveness to ionizing radiation.

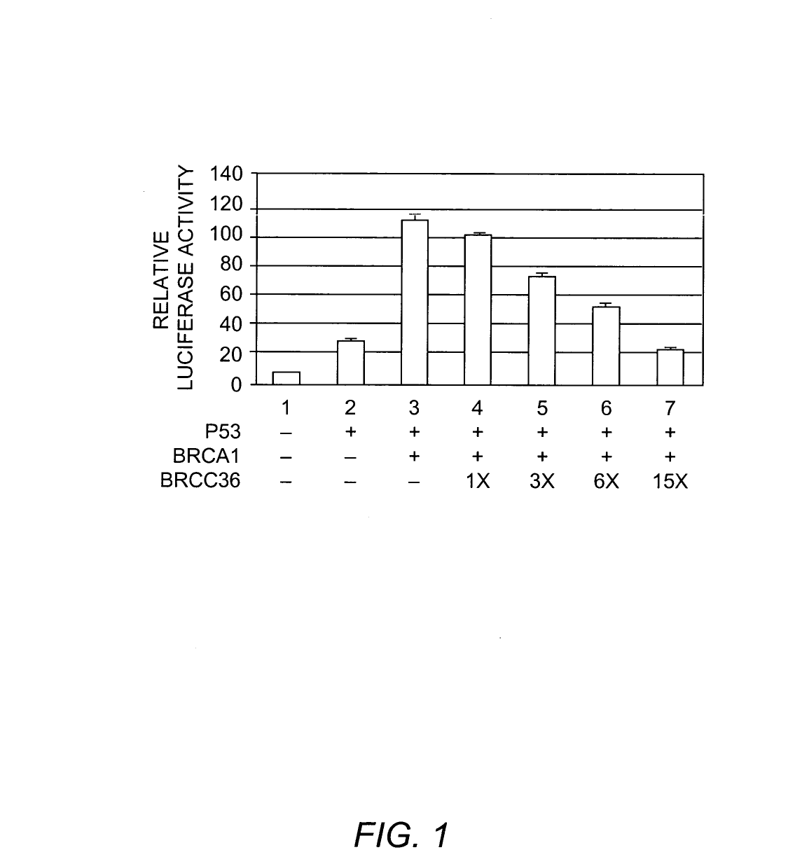

BRCA1 selectively coactivates p53 towards genes that direct DNA repair and cell cycle arrest (MacLachlan, et al. (2002) Mol. Cell. Biol. 22(12):4280-92). Thus, it was determined whether BRCC36 regulates the activation of p53 via BRCA1. The results indicated that BRCC36 inhibited BRCA1 transcriptional activation of p53 in a dose-dependent manner (FIG. 1).

It has now been shown that BRCA1 and BRCA2 are part of a multiprotein complex termed BRCC. A number of novel components of BRCC have been described including BRCC36 (SEQ ID NO:9), a protein with sequence homology to a subunit of the signalosome and proteasome complexes, and BRCC45/BRE (SEQ ID NO:8), a protein that is enriched in the brain and the reproductive organs. Further associated with BRCC is BRCC140 which shares homology with ubiquitin hydrolases. Detailed analysis of these new components of the complex revealed that, similar to BRCA1, depletion of BRCC36 and BRCC45/BRE resulted in increased sensitivity to ionizing radiation and loss of G2/M checkpoint arrest. Further, BRCC36 inhibits the ubiquitin E3 ligase activity and transcriptional regulator activity of BRCC and BRCC140 may function to deubiquitinate BRCC substrates. Moreover, cancer-causing truncations of BRCA1 destabilize the complex and abrogate the association of BRCC36 and BRCC45/BRE with BRCC. These findings identify BRCC, a complex containing hereditary breast cancer susceptibility genes mediating cellular responsiveness to DNA damage. Accordingly, one aspect of the present invention relates to a method of regulating the activity of at least one component of BRCC via an agent which alters the expression or activity of a BRCC36 or BRE subunit of BRCC. The method involves contacting BRCC or a cell containing BRCC with an agent that interacts with a nucleic acid sequence encoding BRCC36 or BRE, or a product thereof, so that the level of expression or activity of BRCC36 or BRE is altered thereby modulating an activity of at least one component of a BRCC. As used herein, a product of a nucleic acid sequence encoding BRCC36 or BRE is intended to include the BRCC36 or BRE mRNA transcript and BRCC36 or BRE protein.

As used herein, regulating an activity of at least one component of a BRCC is intended to include increasing or stimulating as well as decreasing or inhibiting the activity associated with a BRCC component(s). Such activities include, but are not limited to, ubiquitin E3 ligase activity, ubiquitin hydrolase activity, DNA repair activity, and transcriptional regulator activity. A change in an activity of a BRCC component(s) may be determined as exemplified herein or using other methods well-known to those of skill in the art.

It is contemplated that a change in an activity of a BRCC components by an agent which alters the expression or activity of BRCC36 or BRE may be determined using a cell-free or cell-based assay. Such assays containing BRCC are intended to include assays which encompass two or more BRCC components. BRCC components include, but are not limited to, BRCA2 (SEQ ID NO:1), BRCA1 (SEQ ID NO:2), RAD51 (SEQ ID NO:3), BRCC300 (SEQ ID NO:4), BRCC140 (SEQ ID NO:5), BRCC130 (SEQ ID NO:6), BRCA1 Δ11 (SEQ ID NO:7), BRCC80 (SEQ ID NO:8), BRE (SEQ ID NO:9), BRCC36 (SEQ ID NO:10), and BARD1 (SEQ ID NO:11). For example, an in vitro ubiquitination assay may be conducted using BRCC components BRCA1, BARD1 and BRCC36. Further, a cell-based assay for DNA repair activity may be conducted by overexpressing, for example, BARD1 or BRCC36 in a cell and determining cell survival rates upon exposure to ionizing radiation in the presence or absence of a test agent. Preferably, an assay to determine a change in an activity of a BRCC component(s) contains BRCA1, BRCA2 or BRCA1/BRCA2 in a complex with BRCC36 or BRE.

Cell-free assays of the invention may be carried out with proteins which have been recombinantly-produced or chemically-synthesized using conventional methods well-known to the skilled artisan. As will be appreciated by one of skill in the art, a full-length BRCC component protein may be produced for the assays of the invention, however, fragments of a BRCC component protein may also be used provided the fragment maintains the desired binding interaction or activity of the full-length protein (e.g., BRCA truncations of 502-802 and 758-1064). It is also contemplated that it may be desirable to produce a BRCC component protein which binds to the BRCC complex but lacks one or more activities. For example, a BRCC36 protein may be produced which binds BRCC and inhibits the ubiquitin E3 ligase activity but lacks the ability to inhibit transcriptional regulator activity of BRCC.

In general, recombinant production of a BRCC component protein may require incorporation of nucleic acid sequences encoding said protein into a recombinant expression vector in a form suitable for expression of the protein in a host cell. Exemplary nucleic acid sequences include, but are not limited to, those encoding BRCA2 (SEQ ID NO:12), BRCA1 (SEQ ID NO:13), RAD51 (SEQ ID NO:14), BRCC300 (SEQ ID NO:15), BRCC140 (SEQ ID NO:16), BRCC130 (SEQ ID NO:17), BRCA1 Δ11 (SEQ ID NO:18), BRCC80 (SEQ ID NO:19), BRE (SEQ ID NO:20), BRCC36 (SEQ ID NO:21), and BARD1 (SEQ ID NO:22). A suitable form for expression provides that the recombinant expression vector includes one or more regulatory sequences operatively-linked to the nucleic acids encoding the a BRCC component protein in a manner which allows for transcription of the nucleic acids into mRNA and translation of the mRNA into the protein. Regulatory sequences may include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector may depend on such factors as the choice of the host cell to be transfected and/or the level of expression required. Nucleic acid sequences or expression vectors harboring nucleic acid sequences encoding a BRCC component protein may be introduced into a host cell, which may be of eukaryotic or prokaryotic origin, by standard techniques for transforming cells. Suitable methods for transforming host cells may be found in Sambrook, et al. (Molecular Cloning: A Laboratory Manual, 3rd Edition, Cold Spring Harbor Laboratory Press (2000)) and other laboratory manuals. The number of host cells transformed with a nucleic acid sequence encoding a BRCC component protein will depend, at least in part, upon the type of recombinant expression vector used and the type of transformation technique used. Nucleic acids may be introduced into a host cell transiently, or more typically, for long-term expression of a BRCC component protein the nucleic acid sequence is stably integrated into the genome of the host cell or remains as a stable episome in the host cell.

Further, nucleic acid sequences encoding a BRCC component protein may be transferred into a fertilized oocyte of a non-human animal to create a transgenic animal which expresses a BRCC component protein in one or more cell-types. A transgenic animal is an animal having cells that contain a transgene, wherein the transgene was introduced into the animal or an ancestor of the animal at a prenatal, e.g., an embryonic, stage. A transgene is a DNA which is integrated into the genome of a cell from which a transgenic animal develops and which remains in the genome of the mature animal, thereby directing the expression of an encoded gene product in one or more cell-types or tissues of the transgenic animal. Exemplary examples of non-human animals include, but are not limited to, mice, goats, sheep, pigs, cows or other domestic farm animals. Such transgenic animals are useful, for example, for large-scale production of a BRCC component protein (gene pharming) or for basic research investigations.

A transgenic non-human animal may be created, for example, by introducing a nucleic acid sequence encoding a BRCC component protein, typically linked to appropriate regulatory sequences, such as a constitutive or tissue-specific enhancer, into the male pronuclei of a fertilized oocyte, e.g., by microinjection, and allowing the oocyte to develop in a pseudopregnant female foster animal. Intron sequences and polyadenylation signals may also be included in the transgene to increase the efficiency of expression of the transgene. Methods for generating transgenic animals, particularly animals such as mice, have become conventional in the art and are described, for example, in U.S. Pat. Nos. 4,736,866 and 4,870,009. A transgenic founder animal may be used to breed additional animals carrying the transgene. Transgenic animals carrying a transgene encoding a BRCC component protein may further be bred to other transgenic animals carrying other transgenes, e.g., a transgenic animal overexpressing BRCA2 may be bred with a transgenic animal overexpressing BRCC36.

Once produced, a BRCC component protein may be recovered from culture medium or milk as a secreted polypeptide, although it also may be recovered from host cell lysates when directly expressed without a secretory signal. When a BRCC component protein is expressed in a recombinant cell other than one of human origin, the BRCC component protein is substantially free of proteins or polypeptides of human origin. However, it may be necessary to purify the BRCC component protein from recombinant cell proteins or polypeptides using conventional protein purification methods to obtain preparations that are substantially homogeneous as to the BRCC component protein.

A host cell transformed with nucleic acid sequences encoding a BRCC component protein may be used for expressing a BRCC component protein for protein production or may be used in cell-based screening assays of the invention to identify agents which modulate an activity of at least one component of BRCC. Further, a host cell transformed with nucleic acid sequences encoding a BRCC component protein may be transformed with one or more nucleic acid sequences which serve as substrates or targets of BRCC.

In a cell-based assay of the invention, one or more component of BRCC may be endogenous, overexpressed, recombinantly produced or have decreased expression. Accordingly, a cell containing BRCC, may be a cell, for example, which endogenously encodes all components of BRCC and has been transformed to overexpress a tagged BRCC36 protein. Further, it may advantageous to decrease the expression of one component. For example, expression of BRE may be blocked by gene knockout, antisense RNA, RNAi or siRNA molecules to identify agents which specifically interact with BRCC36 to modulate a cells response to ionizing radiation.

In addition to recombinant production, a BRCC component protein may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer, Boston, Mass.). Various fragments of the BRCC component protein may be chemically-synthesized separately and combined using chemical methods to produce a full-length molecule.

Whether recombinantly-produced or chemically-synthesized, a BRCC component protein may be further modified for use in the assays of the invention. For example, the peptides may be glycosylated, phosphorylated or tagged (e.g., fluorescent, FLAG, and the like) using well-known methods.

Screening assays of the invention may be performed in any format that allows rapid preparation and processing of multiple reactions such as in, for example, multi-well plates of the 96-well variety. Stock solutions of the agents as well as assay components are prepared manually and all subsequent pipeting, diluting, mixing, washing, incubating, sample readout and data collecting is done using commercially available robotic pipeting equipment, automated work stations, and analytical instruments for detecting the signal generated by the assay.

In addition to components of BRCC, a variety of other reagents may be included in the screening assays. These include reagents like salts, substrates (e.g., p53 for ubiquitination assays), neutral proteins, e.g., albumin, detergents, etc. which may be used to facilitate optimal protein-protein binding and/or reduce non-specific or background interactions. Also, reagents that otherwise improve the efficiency of the assay, such as protease inhibitors, nuclease inhibitors, anti-microbial agents, and the like may be used. The mixture of components may be added in any order that provides for the requisite binding.

In a screening assay of the invention, an agent may interact with a nucleic acid sequence encoding BRCC36 or BRE, or a product thereof (i.e., mRNA or protein), thereby increasing or decreasing expression of the BRCC36 or BRE protein or increasing or decreasing the activity of the BRCC36 or BRE protein. An activity of BRCC36 or BRE is intended to include a binding interaction with another component of BRCC as well as any enzymatic or catalytic activity of the protein. Preferably, agents will decrease, interfere with or inhibit BRCC36 gene expression or decrease, interfere with or inhibit the activity of BRCC36 protein thereby increasing BRCC activities described herein. Agents which inhibit the expression of BRCC36 or BRE may be identified using the steps of contacting a cell expressing BRCC36 or BRE protein with a test agent and monitoring or measuring the ability of the agent to inhibit or decrease the expression of BRCC36 or BRE. In such an assay, one may measure the expression levels of BRCC36 or BRE using such methods as northern blot analysis, reverse transcriptase PCR, or most preferably by operably linking the promoter or coding region of BRCC36 or BRE to a detectable marker protein such as luciferase, GFP, or β-galactosidase to more easily detect changes in expression levels of BRCC36 or BRE in the presence of a test agent.

Examples of agents which interact with nucleic acid sequences encoding BRCC36 or BRE include, but are not limited to, antisense molecules, interference RNA or ribozymes targeted to BRCC36 or BRE which inhibit the expression or small organic molecules or peptides which are capable of inhibiting expression of BRCC36 or BRE (e.g., by binding to the promoter region of the gene to inhibit transcription and subsequent expression).

Accordingly, one preferred embodiment of the present invention provides antisense, siRNA, or RNAi molecules or ribozymes targeted to nucleic acid sequences encoding BRCC36 or BRE. Using nucleic acid sequences encoding BRCC36 (SEQ ID NO:9) or BRE (SEQ ID NO:8), or homologs, analogs, or alleles thereof, one of skill in the art can readily obtain the corresponding antisense strands of the BRCC36 or BRE sequences. Such antisense sequences or biologically active fragments thereof, capable of hybridizing under stringent conditions (see, J. Sambrook et al, Molecular Cloning: A Laboratory Manual, 2d ed., Cold Spring Harbor Laboratory (1989)) to nucleic acid sequences encoding BRCC36 or BRE, would be useful in inhibiting the expression of BRCC36 or BRE protein, respectively. An example of a highly stringent hybridization condition is hybridization at 2×SSC at 65° C., followed by washing in 0.1×SSC at 65° C. for an hour. Alternatively, an exemplary highly stringent hybridization condition is in 50% formamide, 4×SSC at 42° C. Moderately high stringency conditions may also prove useful, e.g. hybridization in 4×SSC at 55° C., followed by washing in 0.1×SSC at 37° C. for an hour. An alternative exemplary moderately high stringency hybridization condition is in 50% formamide, 4×SSC at 30° C.

RNA, RNAi, siRNA and the like to decrease the expression of BRCC36 or BRE may be specific for sequences in the 5′, 3′ or middle of the mRNA encoding BRCC36 or BRE. The target region may be selected experimentally or empirically. For example, siRNA target sites in a gene of interest are selected by identifying an AA dinucleotide sequence preferably in the coding region and most preferably not near the start codon (within 75 bases) as these may be richer in regulatory protein binding sites which may interfere with binding of the siRNA. (see, e.g., Elbashir, et al. (2001) Nature 411: 494-498). The subsequent 19-27 nucleotides 3′ of the AA dinucleotide may be included in the target site and preferably have a G/C content of 30-50%. Exemplary interference RNA molecules which may be used to block the expression of BRCC36 or BRE include, but are not limited to, AA-GAGGAAGGACCGAGUAGAA (SEQ ID NO:24) and AA-GGUGCAGUACGUGAUUCAA (SEQ ID NO:25), respectively.

Other agents which inhibit expression or activity of BRCC36 or BRE may be identified by screening a library of test agents. Agents are obtained from a wide variety of sources including libraries of synthetic or natural compounds. A library may comprise either collections of pure agents or collections of agent mixtures. Examples of pure agents include, but are not limited to, peptides, polypeptides, antibodies, oligonucleotides, carbohydrates, fatty acids, steroids, purines, pyrimidines, lipids, synthetic or semi-synthetic chemicals, and purified natural products, derivatives, structural analogs or combinations thereof. Examples of agent mixtures include, but are not limited to, extracts of prokaryotic or eukaryotic cells and tissues, as well as fermentation broths and cell or tissue culture supernates. In the case of agent mixtures, one may not only identify those crude mixtures that possess the desired activity, but also monitor purification of the active component from the mixture for characterization and development as a therapeutic drug. In particular, the mixture so identified may be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction may be assayed for the desired activity using the original assay until a pure, biologically active agent is obtained.

Agents of interest in the present invention are those with functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups.

Further, the use of replicable genetic packages, such as the bacteriophages, is one method of generating novel protein entities for regulating BRCC36 or BRE expression or activity. This method generally consists of introducing novel, exogenous DNA segments into the genome of a bacteriophage, or other amplifiable genetic package, so that the protein encoded by the non-native DNA appears on the surface of the phage. When the inserted DNA contains sequence diversity, then each recipient phage displays one variant of the template (parental) amino acid sequence encoded by the DNA, and the phage population (library) displays a vast number of different, but related, amino acid sequences.

Such techniques make it possible not only to screen a large number of potential binding molecules but make it practical to repeat the binding/elution cycles and to build secondary, biased libraries for screening analog-displaying packages that meet initial criteria.

It is well-known to those skilled in the art that it is possible to replace peptides with peptidomimetics. Peptidomimetics are generally preferable as therapeutic agents to peptides owing to their enhanced bioavailability and relative lack of attack from proteolytic enzymes. Accordingly, the present invention also provides peptidomimetics and other lead compounds which can be identified based on data obtained from structural analysis of BRCC36 or BRE. For example, peptide derivatives of a BRCC component protein which interacts with BRCC36 or BRE may be designed to modulate the interaction between BRCC36 or BRE and one or more BRCC component. A potential analog may be examined by computer modeling using a docking program such as GRAM, DOCK, or AUTODOCK. This procedure can include computer fitting of potential analogs. Computer programs can also be employed to estimate the attraction, repulsion, and steric hindrance of an analog to a potential binding site. Generally the tighter the fit (e.g., the lower the steric hindrance, and/or the greater the attractive force) the more potent the potential drug will be since these properties are consistent with a tighter binding constant. Furthermore, the more specificity in the design of a potential drug the more likely that the drug will not interfere with other properties of BRCC36 or BRE expression or activity, e.g., BRCC36 or BRE interactions which are not associated with components of BRCC. This will minimize potential side-effects due to unwanted interactions with other proteins.

Initially a potential analog could be obtained by screening a random peptide library produced by a recombinant bacteriophage, for example, or a chemical library. An analog ligand selected in this manner could be then be systematically modified by computer modeling programs until one or more promising potential ligands are identified.

Such computer modeling allows the selection of a finite number of rational chemical modifications, as opposed to the countless number of essentially random chemical modifications that could be made, and of which any one might lead to a useful drug. Thus, the three-dimensional structure and computer modeling, a large number of compounds may be rapidly screened and a few likely candidates may be determined without the laborious synthesis of untold numbers of compounds.

Once a potential peptidomimetic or lead compound is identified it can be either selected from a library of chemicals commercially available from most large chemical companies including Merck, GlaxoWelcome, Bristol-Meyers Squibb, Monsanto/Searle, Eli Lilly, Novartis and Pharmacia UpJohn, or alternatively the potential ligand is synthesized de novo. The de novo synthesis of one or even a relatively small group of specific compounds is reasonable in the art of drug design.

Agents for modulating activities associated with BRCC may also be found which act on BRCC proteins which interact with or act independent of BRCC36 or BRE. Such agents may be identified from the sources provided herein using assays for detecting the E3 ubiquitin ligase activity, ubiquitin hydrolase activity, DNA repair activity or transcriptional regulator activity of BRCC.

In a preferred embodiment, a method of identifying an agent which modulates an ubiquitin E3 ligase or ubiquitin hydrolase activity of BRCC may involve the steps of contacting BRCC with a test agent and monitoring or measuring the ability of said agent to alter the level of ubiquitination of a select protein which is indicative of ubiquitin E3 ligase activity or ubiquitin hydrolase activity of BRCC. A select protein is defined as a protein which is selectively ubiquinated or deubiquitinated by BRCC. In a preferred embodiment, a select protein is p53. The step of monitoring the ability of the agent to alter the level of ubiquitination of a select protein, such as p53 may be carried out to using an in vitro ubiquitination assay in the presence of ubiquitin (see, e.g., Hashizume, et al. (2001) J. Biol. Chem. 276:14537-14540; Chen, et al. (2002) J. Biol. Chem. 277:22085-22092) or a deubiquitination assay (see, e.g., Gewies and Grimm (2003) Cancer Res. 63(3):682-8; Strayhorn and Wadzinski (2002) Arch. Biochem. Biophys. 400(1):76-84.

In another preferred embodiment, a method of identifying an agent which modulates a DNA repair activity of BRCC may involve the steps of contacting a cell containing BRCC with a test agent and monitoring or measuring the ability of said agent to alter cell survival rates in the presence of ionizing radiation or alter homology-directed DNA repair which is indicative of DNA repair activity of BRCC. The step of monitoring the ability of the agent to alter cell survival rates may carried out using well-established methods (see, e.g., Gowen, et al. (1998) Science 281:1009-1012). Likewise, well-established methods may be used to monitor homology-directed DNA repair (see, e.g., Moynahan, et al. (1999) Mol. Cell 4:511-518). Such an assay may be conducted in vivo by measuring gene conversion repair of an introduced double-strand break. The assay may use the direct repeat-green fluorescence protein (DR-GFP) reporter, which is composed of two mutant GFP genes oriented as a direct repeat. The cleavage site for the rare cutting endonuclease I-SceI mutates one of the GFP repeats. Expression of I-SceI protein in cells that have the DR-GFP substrate integrated into their genome results in a double-strand break in the chromosome at the position of the I-SceI site. Homologous repair by gene conversion reconstructs a functional GFP+ gene. To assay the effect of an agent which inhibits DNA repair activity via homology-directed DNA repair, cells expressing I-SceI are incubated in the presence and absence of an agent and analyzed by flow cytometry either 48 or 96 hour after exposure to the agent. If the agent reduces homology-directed DNA repair activity, there will be a reduction in cell counts compared to cells not exposed to the agent.

In a further preferred embodiment, a method of identifying an agent which modulates a transcriptional regulator activity of BRCC may involve the steps of contacting a cell containing BRCC with a test agent and monitoring or measuring the ability of said agent to alter the expression of genes containing p53 response elements which is indicative of transcriptional regulator activity of BRCC. The step of monitoring the ability of the agent to alter the expression of genes containing p53 response elements may be carried out using well-known methods (see, e.g., Ouchi, et al. (1998) Proc. Natl. Acad. Sci. USA 95:2302-2306).

Other aspects of the present invention relate to agents identified as regulators or modulators of BRCC activities or as regulators or modulators of the expression or activity of BRCC36 or BRE and methods for using these agents to regulate the expression or activity of BRCC36 or BRE thereby modulating an activity of BRCC. These agents may be incorporated into a pharmaceutical composition and administered in an effective amount to a patient having cancer a cancer associated with BRCC. A cancer associated with BRCC is one which originates or is advanced by a mutation or activity of BRCC. Cancers which may be associated with BRCC include, but are not limited to, breast, ovarian, prostate, colon cancers and the like.

An effective amount of an agent which regulates or modulates the expression or activity of BRCC36 or BRE or activity of BRCC is an amount which prevents, eliminates or alleviates at least one sign or symptom of a cancer associated with BRCC. Signs or symptoms of a cancer associated with BRCC vary with the cancer being prevented or treated and are well-known to the skilled clinician. Examples of signs and/or symptoms of a cancer associated with BRCC may include, but are not limited to, tumor size, feelings of weakness, and pain perception. The amount of the agent required to achieve the desired outcome of preventing, eliminating or alleviating a sign or symptom of a cancer associated with BRCC will be dependent on the pharmaceutical composition of the agent, the patient and the condition of the patient, the mode of administration, and the type of condition or disease being prevented or treated.

Pharmaceutical compositions of the present invention comprise an effective amount of an agent which inhibits the expression or activity of BRCC36 or BRE protein or a BRCC activity and a pharmaceutically acceptable carrier. A pharmaceutically acceptable carrier is a material useful for the purpose of administering the medicament, which is preferably sterile and non-toxic, and may be solid, liquid, or gaseous materials, which is otherwise inert and medically acceptable, and is compatible with the active ingredients. A generally recognized compendium of methods and ingredients of pharmaceutical compositions is Remington: The Science and Practice of Pharmacy, Alfonso R. Gennaro, editor, 20th ed. Lippincott Williams & Wilkins: Philadelphia, Pa., 2000.

A pharmaceutical composition may contain other active ingredients such as preservatives. A pharmaceutical composition may take the form of a solution, emulsion, suspension, ointment, cream, granule, powder, drops, spray, tablet, capsule, sachet, lozenge, ampoule, pessary, or suppository. It may be administered by continuous or intermittent infusion, parenterally, intramuscularly, subcutaneously, intravenously, intra-arterially, intrathecally, intraarticularly, transdermally, orally, bucally, intranasally, as a suppository or pessary, topically, as an aerosol, spray, or drops, depending upon whether the preparation is used to treat an internal or external condition or disease. Such administration may be accompanied by pharmacologic studies to determine the optimal dose and schedule and would be within the skill of the ordinary practitioner.

A further aspect of the present invention encompasses antibodies that specifically bind to BRCC36 or BRE or homologs thereof (i.e., antibodies which bind to a single antigenic site or epitope on BRCC36 or BRE). These antibodies are useful for a variety of diagnostic purposes, e.g. diagnosing a disease or condition associated with BRCC. Furthermore, antibodies to BRCC36 or BRE may be used as antagonistic or agonist agents for regulating the activity of BRCC36 or BRE.

Antibodies to BRCC36 or BRE may be generated using methods that are well-known in the art. Such antibodies may include, but are not limited to, polyclonal, monoclonal, chimeric, single chain, Fab fragments, and fragments produced by a Fab expression library. Neutralizing antibodies are especially preferred for therapeutic use.

For the production of antibodies, various hosts including goats, rabbits, rats, mice, humans, and others, may be immunized by injection with BRCC36 or BRE or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to BRCC36 or BRE have an amino acid sequence consisting of at least five amino acids and more preferably at least 10 amino acids. It is also preferable that they are identical to a portion of the amino acid sequence of the natural protein, and they may contain the entire amino acid sequence of a small, naturally occurring molecule. Short stretches of BRCC36 or BRE amino acids may be fused with those of another protein such as keyhole limpet hemocyanin and antibody produced against the chimeric molecule.

Monoclonal antibodies to BRCC36 or BRE may be prepared using any technique which provides for the production of antibody molecules by continuous cell lines in culture. These include, but are not limited to, the hybridoma technique, the human B-cell hybridoma technique, and the EBV-hybridoma technique (Kohler, et al. (1975) Nature 256:495-497; Kozbor, et al. (1985) J. Immunol. Methods 81:31-42; Cote, et al. (1983) Proc. Natl. Acad. Sci. 80:2026-2030; Cole, et al. (1984) Mol. Cell Biol. 62:109-120).

In addition, techniques developed for the production of humanized and chimeric antibodies, the splicing of mouse antibody genes to human antibody genes to obtain a molecule with appropriate antigen specificity and biological activity may be used (Morrison, et al. (1984) Proc. Natl. Acad. Sci. 81, 6851-6855; Neuberger, et al. (1984) Nature 312:604-608; Takeda, et al. (1985) Nature 314:452-454). Alternatively, techniques described for the production of single chain antibodies may be adapted, using methods known in the art, to produce BRCC36- or BRE-specific single chain antibodies. Antibodies with related specificity, but of distinct idiotypic composition, may be generated by chain shuffling from random combinatorial immunoglobulin libraries (Burton (1991) Proc. Natl. Acad. Sci. 88,11120-11123).

Antibodies may also be produced by inducing in vivo production in the lymphocyte population or by screening immunoglobulin libraries or panels of highly specific binding reagents as is well-known in the art (Orlandi, et al. (1989) Proc. Natl. Acad. Sci. 86: 3833-3837; Winter, et al. (1991) Nature 349:293-299).

Antibody fragments, which contain specific binding sites for BRCC36 or BRE, may also be generated. For example, such fragments include, but are not limited to, the F(ab′)₂ fragments which may be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse, et al. (1989) Science 254:1275-1281).

Further provided by the present invention are anti-idiotype antibodies (Ab2) and anti-anti-idiotype antibodies (Ab3). Ab2 are specific for the target to which anti-BRCC36 or anti-BRE antibodies of the invention bind and Ab3 are similar to BRCC36 or BRE antibodies (Ab1) in their binding specificities and biological activities (see, e.g., Wettendorff, et al., “Modulation of anti-tumor immunity by anti-idiotypic antibodies.” In: Idiotypic Network and Diseases, ed. by J. Cerny and J. Hiernaux J, Am. Soc. Microbiol., Washington D.C.: pp. 203-229, (1990)). These anti-idiotype and anti-anti-idiotype antibodies may be produced using techniques well-known to those of skill in the art. For example, an anti-idiotype antibodies (Ab2) of BRCC36 may bear the internal image of BRCC36 and bind to BRCA1 in much the same manner as BRCC36, and thus be useful for the same purposes as BRCC36.

In general, polyclonal antisera, monoclonal antibodies and other antibodies which bind to BRCC36 or BRE as the antigen (Ab1) are useful to identify epitopes of BRCC36 or BRE, to separate BRCC36 or BRE from contaminants in living tissue (e.g., in chromatographic columns and the like), and in general as research tools and as starting material essential for the development of other types of antibodies described above. Anti-idiotype antibodies (Ab2) are useful for binding BRCC and thus may be used in the treatment of cancers. The Ab3 antibodies may be useful for the same reason the Ab1 are useful. Other uses as research tools and as components for separation of BRCC36 or BRE from other contaminants of living tissue, for example, are also contemplated for the above-described antibodies.

Various immunoassays may be used for screening to identify antibodies having the desired specificity. Numerous protocols for competitive binding or immunoradiometric assays using either polyclonal or monoclonal antibodies with established specificity are well known in the art. Such immunoassays typically involve the measurement of complex formation between a specific antibody and BRCC36 or BRE. A two-site, monoclonal-based immunoassay utilizing monoclonal antibodies reactive to two non-interfering BRCC36 or BRE epitopes is preferred, but a competitive binding assay may also be employed (Maddox, supra).

Antibodies may be conjugated to a solid support suitable for a diagnostic assay (e.g., beads, plates, slides or wells formed from materials such as latex or polystyrene) in accordance with known techniques, such as precipitation. Antibodies may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

The present invention further relates to a method for detecting or diagnosing a cancer or the risk of developing a cancer associated with BRCC. The method involves detecting the level or sequence of a nucleic acid sequence encoding BRCC36 or BRE, or a product thereof, in a sample and comparing said level or sequence of a nucleic acid sequence encoding BRCC36 or BRE, or a product thereof, in the sample to a level or sequence of a nucleic acid sequence encoding BRCC36 or BRE, or a product thereof, in a control. In accordance with the method of the invention, one or more identifiable mutations in nucleic acid sequences or proteins of BRCC36 or BRE which correlate with the presence of a BRCC associated cancer should be identified. Mutations in the nucleic acid sequence encoding BRCC36 or BRE may be readily identified using well-known methods. For example, a sample, including, but not limited to, blood or biopsy tissue, may be isolated from an individual having or suspected of having a cancer associated with BRCC and who may exhibit one or more of the typical signs or symptoms associated with the cancer. The nucleic acid sequence of BRCC36 or BRE isolated from the sample is compared to the nucleic acid sequence from a control, e.g., an individual who is known to not have a cancer associated with BRCC, to identify particular changes or mutations (e.g., deletions, insertions, conversions, or point mutations) which may result in aberrant gene expression or protein function which leads to the cancer associated with BRCC.

When detecting mutations in a nucleic acid sequence encoding BRCC36 or BRE in an individual having, suspected of having or at risk of having a BRCC associated cancer, it may be desirable to sequence the genomic locus encoding BRCC36 or BRE or PCR amplify the full-length nucleic acid sequence encoding BRCC36 or BRE or one or more sections of nucleic acid sequence encoding BRCC36 or BRE. Any suitable pair of PCR primers may be used to amplify the these nucleic acid sequence encoding BRCC36 or BRE and would be readily selected by one of skill in the art.

PCR amplification of a full-length nucleic acid sequence encoding BRCC36 or BRE or one or more sections of nucleic acid sequence encoding BRCC36 or BRE may be carried out using any standard PCR reaction reagents and conditions. Various factors such as temperature, magnesium ion concentration, DNA polymerase concentration and dNTP concentration may be considered for a suitable replication fidelity and reaction rate. Moreover, to reduce the likelihood of introducing artifactual mutations as a result of PCR amplification, a proofreading DNA polymerase such as Pho, Taq, Tth, ES4, VENT, DEEPVENT, PFUTurbo, or AmpliTaq polymerase may be used. A detailed discussion of PCR amplification is provided by Eckert, et al., in PCR: A Practical Approach, McPherson, Quirke, and Taylor, eds., IRL Press, Oxford, 1991, pp. 225-244.

PCR amplicons may be sequenced to detect mutations or analyzed by duplex formation with one or more control nucleic acid sequences encoding BRCC36 or BRE. The control may be a PCR-amplified section of the BRCC36 or BRE gene or a restriction enzyme digested fragment. Preferably, the control and the PCR-amplified section of nucleic acid sequence encoding BRCC36 or BRE isolated from the sample are similar in length and encompass the same sections of the BRCC36 or BRE gene. In general, PCR products of sections of nucleic acid sequences encoding BRCC36 or BRE from the sample are mixed with PCR products of controls and heat denatured, e.g., heating to 95° C. The mixture is then slowly cooled to allow hybridization to occur between the amplified section of BRCC36 or BRE from the sample and the control, thereby producing duplexes. Duplexes which may be produced include homoduplexes and heteroduplexes. Homoduplexes are double-stranded DNA fragments wherein the strands are fully complementary. Heteroduplexes are double-stranded DNA fragments wherein the strands are not complementary and differ by at least one base pair. If the amplified section of BRCC36 or BRE from the sample contains a mutation and is hybridized to a wild-type control, a heteroduplex will be produced. However, if the amplified section of BRCC36 or BRE from the sample contains a particular mutation which is also present in the control, a homoduplex will be produced. Likewise, an amplified section of BRCC36 or BRE from the sample lacking a mutation will form a homoduplex with a wild-type control.

When analyzing duplexes to detect mutations in nucleic acid sequences encoding BRCC36 or BRE from samples, any suitable method may be employed including, but not limited to, chemical cleavage mismatch detection (CCMD), enzymatic cleavage of mismatches (ECM), degrading gradient gel electrophoresis (DGGE), heteroduplex analysis combined with mutation detection enhancement (HAMDE) gel electrophoresis, or denaturing high performance liquid chromatography (DHPLC). Duplex analysis may also be combined with single-strand conformational polymorphism analysis (SSCP).

Mutations in BRCC36 or BRE protein sequences may also be identified by sequencing BRCC36 or BRE proteins either directly by, e.g., Edman degradation, or indirectly by antibody binding. A collection of monoclonal antibodies which specifically interact with various epitopes of wild-type BRCC36 or BRE may be used to detect sequence changes or variations (e.g., point mutations, deletions, insertions, and the like) by no longer binding to a mutated form of BRCC36 or BRE.

Further, levels of nucleic acid sequences encoding BRCC36 or BRE may be used to determine if an individual has is at risk of having a BRCC associated cancer. Levels of nucleic acid sequences encoding BRCC36 or BRE may be detected using standard methods such as northern blot analysis, RT-PCR, quantitative RT-PCR, real-time PCR, in situ hybridization and the like. Probes and primers useful for such detection methods may be readily obtained using the sequences disclosed herein. Levels of BRCC36 or BRE expression in a sample, such blood or a biopsy sample from an individual having or suspected of having a BRCC associated cancer, may be compared to levels in a control, e.g., blood or biopsy sample from an individual who does not have a cancer associated with BRCC. A change such as an increase or decrease in the levels of BRCC36 or BRE in the sample as compared to the control indicates that the individual from whom the sample was isolated may have or may be at risk at developing or having a cancer associated with BRCC.

Likewise, levels of BRCC36 or BRE protein may be determined by contacting a sample, e.g., a biopsy sample, blood sample, or other cell sample, with a selected BRCC36 or BRE antibody and measuring or detecting the level or presence of BRCC36 or BRE in the sample wherein a change in the presence or level of BRCC36 or BRE as compared to a control indicates the individual from whom the sample was taken has a cancer associated with BRCC or has an increased risk of developing a cancer associated with BRCC. A control may be blood or a biopsy sample from an individual(s) known to not have or be at risk at having a cancer associated with BRCC. As exemplified herein, an increase in BRCC36 expression is indicative of breast cancer.

For use as a diagnostic reagent, a BRCC36 or BRE antibody may optionally be labeled using diagnostic labels, such as radioactive labels, calorimetric enzyme label systems and the like conventionally used in diagnostic or therapeutic methods. The antibodies may be used to measure abnormal BRCC36 or BRE levels in selected mammalian tissue using conventional diagnostic assays, e.g., immunostaining. For example, in biopsies of tumor tissue, increases or losses in BRCC36 or BRE expression in tumor tissue could be directly verified by immunostaining.

It is further contemplated that agents which regulate the activity of BRCC300, BRCC140, BRCC130, or BRAC1 Δ11 may identified using the general methods of the invention and be useful in modulating the activity of BRCC. Moreover, antibodies directed to BRCC300, BRCC140, BRCC130, or BRCA1 Δ11 may be used for diagnosing diseases or conditions associated with BRCC.

### EXAMPLE 1

### Affinity-Purification of Flag-BARD1

Flag-BARD1 and a selectable marker for puromycin resistance were co-transfected into 293 human embryonic kidney cells or H1299 human lung cancer cells. Transfected cells were grown in the presence of 5 μg/ml puromycin, and individual colonies were isolated and analyzed for FLAG®-BARD1 expression. To purify the BRCC complex, nuclear extract from the FLAG®-BARD1 cell line was incubated with anti-FLAG® M2 affinity gel (SIGMA, St. Louis, Mo.). After extensive washing with buffer A (20 mM Tris-HCl pH 7.9, 0.5 M KCl, 10% glycerol, 1 mM EDTA, 5 mM DTT, 0.5% NP40), the affinity column was eluted with buffer A containing FLAG® peptide (400 μg/ml) according to manufacturer's instructions (SIGMA, St. Louis, Mo.). Other FLAG®-containing complexes were purified using a similar procedure to that described for full-length BRAD1. Analysis of BRCC on gel filtration was conducted using well-established methods (Bochar, et al. (2000) Cell 102:257-65).

### EXAMPLE 2

### Ionizing Radiation Treatment

Approximately 2×10⁴ cells were exposed to various doses of IR, and returned to culture for 3-4 days. Cells were trypsinized, stained with trypan blue, and numbers of live cells were counted. Each experiment included three repeats for each dosage and cell line.

### EXAMPLE 3

### In Vitro Ubiquitination Assays

Ubiquitination assays were conducted in a final volume of 30 μl, containing 50 mM Tris-HCl, pH 7.4, 5 mM MgCl₂, 2 mM ATP, 0.6 mM dithiothreitol, 3 μg of ubiquitin, 10 ng of E1, 30 ng of the specified E2, and the indicated amount and type of E3 as specified. p53 was added as a substrate as indicated. The reactions were incubated at 37° C. for 1 hour unless otherwise indicated. Reactions were stopped with 15 μl of 5× SDS loading buffer. Samples were boiled for 3 minutes prior to SDS-PAGE analysis followed by western blot analysis.

For kinetic studies, ubiquitination reactions were setup with 10 ng of GST-BRCA1/FLAG-BARD1 and 18-30 ng of p53 as the substrate. BRCC36 (0 ng, 24 ng or 48 ng) was added for each substrate concentration. The reactions were incubated at 37° C. for 10 min followed by SDS-PAGE analysis and western blot analysis using antibodies against p53. The poly-ubiquitination products of p53 were quantified using NIH Image, and the data were subjected to a Lineweaver-Burk plot.

### EXAMPLE 4

### Protein Identification Using LC-MS/MS

Gel bands were excised from Colloidal Coomassie-stained gels, bands were destained, alkylated with iodoacetamide, and digested using modified Trypsin (Promega, Madison, Wis.) for 16 hours at 37° C. (Speicher, et al. (2000) J. Biomol. Tech. 11:74-86). A portion of the extracted peptides were loaded onto a nanocapillary reverse-phase 75 μm column terminating in a nanospray 15 μm tip (New Objective, Woburn, Mass.) packed with Porous R2 resin (Applied Biosystems, Foster City, Calif.). The nanocolumn was directly coupled to a Thermo Finnigan LCQ quadrupole ion trap mass spectrometer (Thermo Finnigan, San Jose, Calif.) and peptides were eluted into the mass spectrometer using an acetic acid-acetonitrile gradient. Data was acquired using triple play mode to automatically obtain peptide masses, peptide charge states, and MS/MS spectra. The resulting data were searched against the non-redundant NCBI using TurboSEQUEST Browser (Eng, et al. (1994) J. Am. Soc. Mass Spectrom. 5:976-989; Chittum, et al. (1998) Biochemistry 37:10866-10870) to identify proteins.

### EXAMPLE 5

### Phylogenetic Tree Analysis

Phylogenetic tree analysis of protein sequences from JAB/MPN domains (smart00232) were conducted by the Kimura distance method algorithm, using Clustalw and Phylip software (http://bioweb.pasteur.fr/). Accession numbers for the individual proteins used for the analysis are as follows: Homo sapiens C6.1A/BRCC36, accession number P46736; Mus musculus BRCC36, accession number P46737; Arabidopsis thaliana hypothetical protein, accession number AAM10390; H. sapiens PAD1/POH1, accession number AAC51866; M. musculus PAD1, accession number NP_{—}067501; Drosophila melanogaster RPN11, accession number AAF52215; A. thaliana hypothetical protein, accession number BAA7246; Caenorhabditis elegans hypothetical protein, accession number T33344; Schizosaccharomyces pombe PAD1/sks1, accession number P41878; Saccharomyces cerevisiae RPN11, accession number S56259; C. elegans hypothetical protein, accession number P41883; A. thaliana Sub5aCOP9, accession number AAL58104; A. thaliana Sub5bCOP9, accession number AAC36344; H. sapiens Jab1, accession number NP_{—}006828; M. musculus Jab1, accession number AAD03470; D. melanogaster Sub5COP9, accession number AAD28608; and S. pombe Sub5COP9, accession number NP_{—}593131.

### EXAMPLE 6

### Inmmunoblot Analysis

Anti-BRCA1 and anti-BRCA2 antibodies are known in the art (Bochar, et al. (2000) supra; Marmorstein, et al. (2001) Cell 104:247-257). Anti-Rad51 antibodies were commercially obtained (Upstate Biotechnology, Lake Placid, N.Y.). Anti-BRCC36 and anti-BRE antibodies were developed to peptides corresponding to the C-terminal last 20 amino acid residues of each protein. The antibodies were affinity-purified using the cognate peptide. Immunoblotting with alkaline phosphatase was performed using well-known methodologies (Bochar, et al. (2000) supra).

### EXAMPLE 7

### Protein Purification and Protein-Protein Interactions

Recombinant BRCA1 and BARD1 was purified as described (Chen, et al. (2002) J. Biol. Chem. 277:22085-22092). For in vitro interaction studies, whole cell extract from BL21 (GST-BRCA1 (1-639) and GST-BRCA1 fragments 1-6) and BI2J (GST-BRCA1/FLAG®-BARD1) were prepared. 5 μg of purified (His)6-FLAG®-BRCC36 protein were incubated with 400 μg of whole cell extracts or 10 μg of purified GST-BRCA1 fragment in binding buffer (20 mM Tris-HCl pH 7.9, 0.1 M KCl, 10% Glycerol, 1 mM EDTA, 2 mM MgCl 2,5 mM DTT, 0.1% NP40) for 2 hours at 4° C. with inversion. Gluthatione-sepharose beads (Pharmacia, Peapack, N.J.), previously equilibrated in binding buffer, were added to the samples and inverted for an additionnal 2 hours at 4° C. Beads were collected by centrifugation at 2000×g, 30 seconds, washed several times in binding buffer containing 500 mM KCl and 0.1% NP40 and resuspended in 2× SDS loading buffer. Proteins were resolved by SDS-PAGE and western blot analysis was performed using anti-FLAG® antibodies (SIGMA, St. Louis, Mo.).

### EXAMPLE 8

### Plasmid, si RNA and Transfection

BARD1 and BRCC36 were cloned in pFLAG-CMV2 (SIGMA, St. Louis, Mo.) and pCMV-5A (SIGMA, St. Louis, Mo.) vectors, respectively, by using standard PCR techniques. The siRNA molecules were artificially synthesized (Dharmacon, Inc., Lafayette, Colo.). The sequence of the BRCA1 siRNA was AA-CUUAGGUGAAGCAGCAUCU (SEQ ID NO:23); the BRCC36 siRNA was AA-GAGGAAGGACCGAGUAGAA (SEQ ID NO:24); the BRE siRNA was AA-GGUGCAGUACGUGAUUCAA (SEQ ID NO:25) and the control siRNA to transcription factor TFII-I was AA-GUUACUCAGCCAAGAACGA (SEQ ID NO:26). Transfection of the siRNA was performed with LIPOFECTAMINE™ 2000 (Life Technologies, Inc., Carlsbad, Calif.) according to the manufacturer's instructions. Briefly, cells were plated in 10-cm dish to 40% confluence. For each dish, 1.6 nmole siRNA was mixed with 20 μl LIPOFECTAMINE™ 2000 in 3 ml OPTI-MEM®. The mixture was added to cells and incubated for 6 hours. Twenty-four hours later, a second transfection was similarly performed. Cells were treated or harvested as indicated 72 hours after the initial transfection.

### EXAMPLE 9

### Flow Cytometric Analysis of Phosphorylated Histone H3 Staining

Seventy-two hours after siRNA transfection, cells were γ-irradiated at different dosage and then incubated at 37° C. for 1 hour before fixation in 70% ethanol. Cells were stained in 200 μl phosphate-buffered saline (PBS) containing 1% bovine serum albumin and 1 μg of polyclonal antibody against phosphorylated H3 (Upstate Biotechnology, Lake Placid, N.Y.) at 4° C. overnight, and then FITC-conjugated anti-rabbit IgG (Vector Laboratories, Inc., Burlingame, Calif.) at room temperature for 1 hour. After washing with PBS, cells were suspended in 5 μg/ml PI (SIGMA, St. Louis, Mo.), and the cellular fluorescence was measured by a flow cytometer.

What is claimed is:

1. A method for modulating the activity of at least one component of a BRCA1-BRCA2-containing complex (BRCC) comprising contacting a BRCC or cell containing a BRCC with an agent that interacts with a nucleic acid sequence encoding BRCC36 or BRE, or a product thereof, so that the expression or activity of BRCC36 or BRE is altered thereby modulating an activity of at least one component of a BRCC.

2. The method of claim 1 wherein the at least one component of a BRCC comprises BRCA2, BRCA1, RAD51, BARD1, BRCC300, BRCC140, BRCC130, BRCA1 Δ11, BRCC80, BRE, or BRCC36.

3. The method of claim 1 wherein the activity of the component comprises ubiquitin E3 ligase activity, ubiquitin hydrolase activity, DNA repair activity, or transcriptional regulator activity.

4. A method for identifying an agent that modulates the ubiquitin E3 ligase activity or ubiquitin hydrolase activity of BRCC comprising contacting BRCC with a test agent and monitoring the ability of said agent to alter the level of ubiquitination of select protein which is indicative of ubiquitin E3 ligase activity or ubiquitin hydrolase activity of BRCC.

5. A method for identifying an agent that modulates the DNA repair activity of BRCC comprising contacting a cell containing BRCC with a test agent and monitoring the ability of said agent to alter cell survival rates in the presence of ionizing radiation or alter homology-directed DNA repair which is indicative of DNA repair activity of BRCC.

6. A method for identifying an agent that modulates the transcriptional regulator activity of BRCC comprising contacting a cell containing BRCC with a test agent and monitoring the ability of said agent to alter the expression of genes containing p53 response elements which is indicative of transcriptional regulator activity of BRCC.

7. An agent identified by the method of claim 4.

8. An agent identified by the method of claim 5.

9. An agent identified by the method of claim 6.

10. A method for treating a cancer associated with BRCC comprising administering to a patient having a cancer associated with BRCC an effective amount of an agent identified by the method of claim 4.

11. A method for treating a cancer associated with BRCC comprising administering to a patient having a cancer associated with BRCC an effective amount of an agent identified by the method of claim 5.

12. A method for treating a cancer associated with BRCC comprising administering to a patient having a cancer associated with BRCC an effective amount of an agent identified by the method of claim 6.

13. A method for identifying an agent that inhibits the expression of BRCC36 or BRE protein comprising contacting a cell expressing BRCC36 or BRE protein with a test agent and monitoring the ability of said agent to alter the expression of BRCC36 or BRE protein.

14. An agent identified by the method of claim 13.

15. A method for treating a cancer associated with BRCC comprising administering to a patient having a cancer associated with BRCC an effective amount of an agent of claim 14.

16. An antibody which specifically recognizes BRCC36 or BRE protein.

17. A method for diagnosing a cancer or the risk of developing a cancer associated with BRCC comprising
detecting a level or sequence of a nucleic acid sequence encoding BRCC36 or BRE, or a product thereof, in sample;
comparing said level or sequence of a nucleic acid sequence encoding BRCC36 or BRE, or a product thereof in the sample to a level or sequence of a nucleic acid sequence encoding BRCC36 or BRE, or a product thereof, in a control;
wherein a change in the level or sequence of a nucleic acid sequence encoding BRCC36 or BRE, or a product thereof, in the sample as compared to the control is indicative of a cancer or the risk of developing a cancer associated with BRCC.


SEQUENCE LISTING (26 sequences)

SEQ ID NO: 1 - protein, 3418 aa, Homo sapiens [sequence omitted]

SEQ ID NO: 2 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 3 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 4 - protein, 2626 aa, Homo sapiens
MTTELSSYGYLGSENSALFNRVCTSYCEEGVESAALLGCDNSSSTGNTSFSSLLRDYPLHLFHMKTPFPLSFIECPSKSELTSLGIILYFLDDMEDEIFRHYAELRPQNFPCSVRRNNSAFMTSSDFAERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTKVRMQRDLVDTAQRSAPGPSARRRVADMTARGQSPLAPLLETLEDPSASHGGQTDAYLTLTSRMTGEEGKEVITEIEKKLPRLYKVLKVSSIIDSLEILFNKGETHSAVVDFEALNVIVRLIEQAPIQMGEEAVRWAKLVIPLVVHSAQKVHLRGATALEMGMPLLLQKQQEIASITEQLMTTTLHRSGSFINSLLQLEELGFRSGAPMIKKIAFIAWKSLIDNFALNPDILCSAKRLKLLMQPLSSIHVRTETLALTKLEVWWYLLMRLGPHLPANFEQVCVPLIQSTISIDSNASPQGNSCHVATSPGLNPMTPVHKGASSPYGAPGTPRMNLSSNLGGMATIPSIQLLGLEMLLHFLLGPEALSFAKQNKLVLSLEPLEHPLISSPSFFSKHANTLITAVHDSFVAVGKDAPGNKKEKPGSEVLTLLLKSLESIVKSEVFPVSKTLGTPALFLIQLIFNNFLECGVSDERFFLSLESLVGCVLSGPTSPLAFSDSVLNVINQNAKQLENKEHLWKMWSVIVTPLTELINQTNEVNQGDALEHNFSAIYGALTLPVNHIFSEQRFPVATMKTLLRTWSELYRAFARCAALVATAEENLCCEELSSKIMSSLEDEGFSNLLFVDRIIYIITVMVDCIDFSPYNIKYQPKVKSPQRPSDWSKKKNEPLGKLTSLFKLIVKVIYSFHTLSFKEAHSDTLFTIGNSITGIISSVLGHISLPSMIRKIFATLTRPLALFYENSKLDEVPKVYSCLNNKLEKLLGEIIACLQFSYTGTYDSELLEQLSPLLCIIFLHKNKQIRKQSAQFWNATFAKVMMLVYPEELKPVLTQAKQKFLLLLPGLETVEMMEESSGPYSDGLKLESSSLKVKGEILLEEEKSTDFVFIPPEGKDAKERILTDHQKEVLKTKRFEEQMDSDIVIPQDVTEDCGMAEHLEKSSLSNNECGSLDKTSPEMSNSNNDERKKALISSRKTSTECASSTENSFVVSSSSVSNTTVAGTPPYPTSRRQTFITLEKFDGSENRPFSPSPLNNISSTVTVKNNQETMIKTDFLPKAKQREGTFSKSDSEKIVNGTKRSSRRAGKAEQTGNKRSKPLMRSEPEKNTEESVEGIVVLENNPPGLLNQTECVSDNQVHLSESTMEHDNTKLKAATVENAVLLETNTVEEKNVEINLESKENTPPVVISADQMVNEDSQVQITPNQKTLRRSSRRRSEVVESTTESQDKENSHQKKERRKEEEKPLQKSPLHIKDDVLPKQKLIAEQTLQENLIEKGSNLHEKTLGETSANAETEQNKKKADPENIKSEGDGTQDIVDKSSEKLVRGRTRYQTRRASQGLLSSIENSESDSSEAKEEGSRKKRSGKWKNKSNESVDIQDQEEKVVKQECIKAENQSHDYKATSEEDVSIKSPICEKQDESNTVICQDSTVTSDLLQVPDDLPNVCEEKNETSKYAEYSFTSLPVPESNLRTRNAIKRLHKRDSFDNCSLGESSKIGISDISSLSEKTFQTLECQHKRSRRVRRSKGCDCCGEKSQPQEKSLIGLKNTENNDVEISETKKADVQAPVSPSETSQANPYSEGQFLDEHHSVNFHLGLKEDNDTINDSLIVSETKSKENTMQESLPSGIVNFREEICDMDSSEAMSLESQESPNENFKTVGPCLGDSKNVSQESLETKEEKPEETPKMELSLENVTVEGNACKVTESNLEKAKTMELNVGNEASFHGQERTKTGISEEAAIEENKRNDDSEADTAKLNAKEVATEEFNSDISLSDNTTPVKLNAQTEISEQTAAGELDGGNDVSDLHSSEETNTKMKNNEEMMIGEAMAETGHDGETENEGITTKTSKPDEAETNMLTAEMDNFVCDTVEMSTEEGIIDANKTETNTEYSKSEEKLDNNQMVMESDILQEDHHTSQKVEEPSQCLASGTAISELIIEDNNASPQKLRELDPSLVSANDSPSGMQTRCVWSPLASPSTSILKRGLKRSQEDEISSPVNKVRRVSFADPIYQAGLADDIDRRCSIVRSHSSNSSPIGKSVKTSPTTQSKSVDLCVTASEIYSKISEMAKESIPCPTESVYPPLVNCVAPVDIILPQITSNMWARGLGQLIRAKNIKTIGDLSTLTASEIKTLPIRSPKVSNVKKALRIYHEQQVKTRGLEEIPVFDISEKTVNGIENKSLSPDEERLVSDIIDPVALEIPLSKNLLAQISALALQLDSEDLHNYSGSQLFEMHEKLSSEGIFLATVCQFIIVDKAEFTGQQAYMLIEVDALRSDELKRMEKAD

SEQ ID NO: 5 - protein, 1186 aa, Homo sapiens [sequence omitted]

SEQ ID NO: 6 - protein, 982 aa, Homo sapiens
MAPRLQLEKAAWRWAETVRPEEVSQEHIETAYRIWLEPCIRGVCRRNCKGNPNCLVGIGEHIWLGEIDENSFHNIDDPNCERRKKNSFVGLTNLGATCYVNTFLQVWFLNLELRQALYLCPSTCSDYMLGDGIQEEKDYEPQTICEHLQYLFALLQNSNRRYIDPSGFVKALGLDTGQQQDAQEFSKLFMSLLEDTLSKQKNPDVRNIVQQQFCGEYAYVTVCNQCGRESKLLSKFYELELNIQGHKQLTDCISEFLKEEKLEGDNRYFCENCQSKQNATRKIRLLSLPCTLNLQLMRFVFDRQTGHKKKLNTYIGFSEILDMEPYVEHKGGSYVYELSAVLIHRGVSAYSGHYIAHVKDPQSGEWYKFNDEDIEKMEGKKLQLGIEEDLAEPSKSQTRKPKCGKGTHCSRNAYMLVYRLQTQEKPNTTVQVPAFLQELVDRDNSKFEEWCIEMAEMRKQSVDKGKAKHEEVKELYQRLPAGAEPYEFVSLEWLQKWLDESTPTKPIDNHACLCSHDKLHPDKISIMKRISEYAADIFYSRYGGGPRLTVKALCKECVVERCRILRLKNQLNEDYKTVNNLLKAAVKGDGFWVGKSSLRSWRQLALEQLDEQDGDAEQSNGKMNGSTLNKDESKEERKEEEELNFNEDILCPHGELCISENERRLVSKEAWSKLQQYFPKAPEFPSYKECCSQCKILEREGEENEALHKMIANEQKTSLPNLFQDKNRPCLSNWPEDTDVLYIVSQFFVEEWRKFVRKPTRCSPVSSVGNSALLCPHGGLMFTFASMTKEDSKLIALIWPSEWQMIQKLFVVDHVIKITRIEVGDVNPSETQYISEPKLCPECREGLLCQQQRDLREYTQATIYVHKVVDNKKVMKDSAPELNVSSSETEEDKEEAKPDGEKDPDFNQIMHAFSVAPFDQNLSIDGKILSDDCATLGTLGVIPESVILLKADEPIADYAAMDDVMQVCMPEEGFKGTGLLGH

SEQ ID NO: 7 - protein, 721 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 8 - protein, 719 aa, Homo sapiens
MPRRKKKVKEVSESRNLEKKDVETTSSVSVKRKRRLEDAFIVISDSDGEEPKEENGLQKTKTKQSNRAKCLAKRKIAQMTEEEQFALALKMSEQEAREVNSQEEEEEELLRKAIAESLNSCRPSDASATRSRPLATGPSSQSHQEKTTDSGLTEGIWQLVPPSLFKGSHISQGNEAEEREEPWDHTEKTEEEPVSGSSGSWDQSSQPVFENVNVKSFDRCTGHSAEHTQCGKPQESTGRGSAFLKAVQGSGDTSRHCLPTLADAKGLQDTGGTVNYFWGIPFCPDGVDPNQYTKVILCQLEVYQKSLKMAQRQLLNKKGFGEPVLPRPPSLIQNECGQGEQASEKNGCISEDMGDEDKEERQESRASDWHSKTKDFQESSIKSLKEKLLLEEEPTTSHGQSSQGIVEETSEEGNSVPASQSVAALTSKRSLVLMPESSAEEITVCPETQLSSSETFDLEREVSPGSRDILDGVRIIMADKEVGNKEDAEKEVAISTFSSSNQVSCPLCDQCFPPTKIGRHAMYCNGLMEEDTVLTRRQKEAKTKSDSGTAAQTSLDIDKNEKCYLCKSLVPFREYQCHVDSCLQLAKADQGDGPEGSGRACSTVEGKWQQRLKNPKEKGHSEGRLLSFLEQSEHKTSDADIKSSETGAFRVPSPGMEEAGCSREMQSSFTRRDLNESPVKSFVSISEATDCLVDFKKQVTVQPGSRTRTKAGRGRRRKF

SEQ ID NO: 9 - protein, 415 aa, Homo sapiens
MSPEVALNRISPMLSPFISSVVRNGKVGLDATNCLRITDLKSGCTSLTPGPNCDRFKLHIPYAGETLKWDIIFNAQYPELPPDFIFGEDAEFLPDPSALQNLASWNPSNPECLLLVVKELVQQYHQFQCSRLRESSRLMFEYQTLLEEPQYGENMEIYAGKKNNWTGEFSARFLLKLPVDFSNIPTYLLKDVNEDPGEDVALLSVSFEDTEATQVYPKLYLSPRIEHALGGSSALHIPAFPGGGCLIDYVPQVCHLLTNKVQYVIQGYHKRREYIAAFLSHFGTGVVEYDAEGFTKLTLLLMWKDFCFLVHIDLPLFFPRDQPTLTFQSVYHFTNSGQLYSQAQKNYPYSPRWDGNEMAKRAKGCQGSRDACSPWEQVLAFAVAKTGCKLLQPQRNWPSSRGPPWRASEGERTAQ

SEQ ID NO: 10 - protein, 316 aa, Homo sapiens
MAVQVVQAVQAVHLESDAFLVCLNHALSTEKEEVMGLCIGELNDDTRSDSKFAYTGTEMRTVAEKVDAVRIVHIHSVIILRRSDKRKDRVEISPEQLSAASTEAERLAELTGRPMRVVGWYHSHPHITVWPSHVDVRTQAMYQMMDQGFVGLIFSCFIEDKNTKTGRVLYTCFQSIQAQKSSESLHGPRDFWSSSQHISIEGQKEEERYERIEIPIHIVPHVTIGKVCLESAVELPKILCQEEQDAYRRIHSLTHLDSVTKIHNGSVFTKNLCSQMSAVSGPLLQWLEDRLEQNQQHLQELQQEKEELMQELSSLE

SEQ ID NO: 11 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 12 - DNA, 10987 bp, Homo sapiens
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 13 - DNA, 5711 bp, Homo sapiens
agctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 14 - DNA, 2229 bp, Homo sapiens
ccgcgcgcagcggccagagaccgagccctaaggagagtgcggcgcttcccgaggcgtgcagctgggaactgcaactcatctgggttgtgcgcagaaggctggggcaagcgagtagagaagtggagcgtaagccaggggcgttgggggccgtgcgggtcgggcgcgtgccacgcccgcggggtgaagtcggagcgcggggcctgctggagagaggagcgctgcggaccgagtaatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactgaatcattgggtttttcctctgttaaaaaccttaagtgctgcagcctaatgagagtgcactgctccctggggttctctacaggcctcttcctgttgtgactgccaggataaagcttccgggaaaacagctattatatcagcttttctgatggtataaacaggagacaggtcagtagtcacaaactgatctaaaatgtttattccttctgtagtgtattaatctctgtgtgttttctttggttttggaggaggggtatgaagtatctttgacatggtgccttaggaatgacttgggtttaacaagctgtctactggacaatcttatgtttccaagagaactaaagctggagagacctgacccttctctcacttctaaattaatggtaaaataaaatgcctcagctatgtagcaaagggaatgggtctgcacagattctttttttctgtcagtaaaactctcaagcaggtttttaagttgtctgtctgaatgatcttgtgtaagggtttggttatggagtcttgtgccaaacctactaggccattagcccttcaccatctacctgcttggtctttcattgctaagactaactcaagataatcctagagtcttaaagcatttcaggccagtgtggtgtcttgcgcctgtactcccagcactttgggaggccgaggcaggtggatcgcttgagccaggagttttaagtccagcttggccaagatggtgaaatcccatctctacaaaaaatgcagaacttaatctggacacactgttacacgtgcctgtagtcccagctactctatagcctgaggtgggagaatcacttaagcctggaaggtggaagttgcagtgagtcgagattgcactgctgcattccagccagggtgacagagtgagaccatgtttcaaacaagaaacatttcagagggcaagtaaacagatttgattgtgaggcttctaataaagtagttattagtagtg

SEQ ID NO: 15 - DNA, 7840 bp, Homo sapiens
atgacaactgaattaagttcatatgggtatttgggatccgagaattctgctttgttcaatagagtctgcaccagttactgtgaagaaggagtagagtctgctgccctcttgggatgtgacaatagctcatctactggaaataccagtttctcttcccttctgagggattaccctctgcatcttttccatatgaaaaccccatttcctctttcctttattgaatgcccctcaaaatcagaacttacatcattaggcattattctctatttccttgatgatatggaggatgagattttcagacactatgcagagctgaggccacagaatttcccctgttccgtaagaaggaacaacagtgcttttatgacatcatctgattttgcagaacgagcagccggtgtgtaccacagagaagcacggtctggcaaatacaagctcacctacgcagaagctaaggcggtgtgtgaatttgaaggcggccatctcgcaacttacaagcagctagaggcagccagaaaaattggatttcatgtctgtgctgctggatggatggctaagggcagagttggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcaaatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaaggaaaaaatacaaaagtcagaatgcaacgagatctggttgatactgcacagagatcagctccagggcccagtgcaaggagaagggtggccgacatgacggccaggggtcagagccccctcgcgccgctgttggagactttggaagacccttctgcctcccatggagggcagactgacgcttacctgactctgaccagtcgtatgactggagaagaaggaaaagaagtaattacagaaattgagaaaaaacttcctcggctgtacaaagttttaaaggtatccagtataattgattcattagaaatactgtttaacaaaggagagacgcattctgctgttgttgattttgaagcattaaatgttatcgtaaggctaattgaacaagccccaattcaaatgggagaagaggcagtgaggtgggcaaaactggtcatacctttagtggttcattcagcacaaaaggtacatttgcggggagcaactgctctggagatgggaatgccattattgcttcagaaacagcaagaaatagcatctattacggagcagcttatgactactaccttgcatcgaagtgggagtttcatcaattctctcttgcaactagaagaacttggatttcgtagtggagcacccatgattaaaaagatagcttttattgcttggaagagtttaatagataattttgctttaaatccagatatactatgtagtgcaaaaagactcaagttgttaatgcagcctttgagttccatccatgtgagaacagaaactctagcattaacaaaactagaagtctggtggtatttactgatgagacttggacctcatcttcctgctaattttgaacaggtttgtgtgcctctgattcaaagtacaataagcattgattctaatgcctcacctcagggcaattcgtgtcatgtagctacatctccaggtttaaatcctatgactcctgtacacaaaggtgcttcctccccgtacggagccccgggaactccccgaatgaacctgagttcgaatttaggtggaatggccacaatcccatccattcaacttttgggacttgaaatgttgcttcatttcttgttgggtccagaagccttgagttttgctaagcaaaataaacttgtgctgagcttagagccattggaacatccgttaatcagcagcccttcctttttttccaaacatgcaaatacacttatcactgctgttcatgatagctttgttgcagttggaaaagatgcccccggtaacaaaaaagagaaaccaggttctgaagttttgactctcttattaaagtctttggaaagcatagtaaagtctgaagtatttcctgtatcaaaaacgctgggaactccagctttgttcttaattcaattaattttcaacaatttcttggaatgtggtgtatcagatgaaaggttctttctcagtttggaatcacttgtaggctgtgttctttctggtccaacttcaccactagctttcagtgactcagttttaaatgttattaatcaaaatgcaaagcagttggaaaataaggagcatctctggaaaatgtggagtgttatagtcaccccattaactgaattgattaatcagaccaatgaagtaaatcaaggtgatgccttagaacataattttagtgccatctatggtgcattgactttaccagtaaaccacattttttcagaacagagatttccagtggccaccatgaagactttgcttagaacttggtcagaattatatagagcatttgctcgttgtgctgctttggtggcaacagcagaagagaacttgtgctgtgaggaactttcttccaagataatgtccagtttggaagatgaaggcttttctaatttgttgttcgtggatagaattatttatattattactgtaatggttgattgcattgacttctcaccatataatattaaatatcagcccaaagttaaatcaccacagagaccttcagattggtccaaaaagaagaatgagcccctagggaaattgacttctttatttaaacttattgtgaaagtgatctattctttccacacactgagcttcaaggaagcacattctgataccctcttcactattggcaactcaatcaccggcattatttccagtgtacttgggcatatttctttgccttctatgatccgaaaaatatttgcaactttaacaagacctctggcattattttatgaaaactcaaagcttgatgaagttcctaaagtatatagttgtctgaacaacaagttagaaaagctactgggagaaattattgcttgtctgcaattcagctacaccggaacttatgatagtgaacttcttgaacaactctccccactattatgcataatatttctgcacaagaataaacagattcgaaaacagagtgctcagttctggaatgccacttttgccaaagtgatgatgttggtttatcctgaagagttaaaaccagtactaacacaagccaaacaaaaatttctgctcctgttgcctggtttggaaactgttgaaatgatggaggaatccagtggaccatattctgatggactgaaacttgaatcttcgtctttaaaagtaaagggtgaaattcttttggaagaggaaaagtctactgactttgtgtttatacctccagaaggaaaagatgcaaaggaaagaatattaactgatcatcaaaaagaagttctcaaaacaaagcggtttgaggagcaaatggacagtgacattgtcattcctcaagatgtcacggaagactgtggtatggctgaacatcttgaaaagtcctccctttcgaataatgagtgtggttctcttgacaaaaccagtccagaaatgtcaaacagtaataatgatgaaagaaaaaaagctttaatttcatcaaggaaaacatcaactgaatgtgcatctagtacagaaaattctttcgttgtcagcagtagttcagtttctaataccactgttgctggaactcccccataccctacaagtcggaggcaaacctttattactttggagaagtttgatggttcagaaaatagaccttttagtccatcccccttgaataatatttcatcaactgttacagtgaaaaataaccaggaaaccatgattaaaacagattttctaccaaaagcaaagcaaagagaagggactttttcaaaatctgattctgaaaaaatagtgaatggaactaagagatcaagccggagagctggtaaagctgaacaaacagggaataaaaggtctaagcccttaatgagatctgagccggagaaaaatactgaggaatctgttgaaggcattgtagtcttagaaaataacccacctggtttgcttaatcaaacagaatgtgtgtcagataatcaggttcatctttctgaatctacaatggagcatgacaatacaaagcttaaagcagcaacagtggaaaatgctgtattattggaaactaatactgtagaggagaaaaatgtagaaattaatttggaatccaaagagaatacacccccagtagtaatatcagcagatcaaatggtaaatgaggatagtcaggttcagataactccaaatcagaaaacccttagacggtcttcaaggcgacgttcagaagtagtagagtctaccactgaaagccaagataaggaaaatagtcatcaaaaaaaggaacgacgtaaggaagaagaaaaacctcttcagaagagtccattgcatataaaagatgatgtgttacctaaacaaaaactgattgctgaacaaactctacaggagaatttaattgagaaaggaagtaatttacatgagaagactcttggggaaactagtgctaatgcagaaactgaacaaaataaaaaaaaggcagaccctgagaacattaagtctgagggggatggtacccaggacattgtagataagtcctctgagaaactagtcagaggccgaacacggtatcaaactagaagagcatctcagggtttgctttccagcattgaaaactcagaatctgatagttcggaggcaaaagaagaaggttctaggaagaagagatctggaaaatggaaaaacaaaagcaatgaaagtgttgacattcaagatcaagaagagaaagtggtgaaacaggaatgtataaaagctgaaaatcagtcacatgattataaagcaacttctgaagaagatgtaagcataaaatctccgatttgcgaaaaacaagatgaaagtaatactgtaatatgtcaggattctacagtaacttcagatttgttgcaagttcctgatgatttaccaaatgtgtgtgaggaaaaaaatgaaactagcaaatatgcagaatattcctttacaagtctacctgtgccagaatcaaatctaaggactagaaatgccattaagagattacataagcgagactcttttgataattgtagtttgggagaatcctcaaaaatagggatatcagatatttcttcgctttcagaaaaaacttttcaaacacttgaatgccaacacaagagaagtaggagggtgaggagatctaaaggttgtgattgctgtggggaaaaatcacaacctcaggaaaagtcactcattgggttaaagaatacagaaaataatgacgtagagattagtgaaacaaaaaaggcagatgtgcaagcacctgtaagcccatcagaaacttctcaagctaatccatattctgaaggacaatttttagatgaacatcatagtgtgaattttcatttgggtctcaaagaggataatgatactattaatgattcattaattgtttctgaaaccaaatcaaaagaaaacactatgcaagaatctcttccttctggaatagtaaactttagagaggaaatttgtgatatggattctagtgaagcaatgtctcttgaaagccaggagtcacctaatgaaaattttaaaactgttggcccgtgtttaggagactcgaaaaatgtttcacaggaatctttggagacaaaagaagaaaaaccagaagaaaccccaaaaatggaactgagtctagagaatgttactgttgaaggaaatgcatgtaaagtaacagaatccaatctagagaaagcaaaaactatggaattgaatgtaggaaatgaagctagctttcatggacaagagagaaccaaaactggtatttctgaagaagcagcaatagaagaaaataaaagaaatgatgactctgaagcagacacagctaaactgaatgccaaagaagtagcaactgaggaatttaattcagatattagtctttctgataatactacacctgtaaaattgaatgctcaaactgagatttctgaacaaacagcagctggggaactagatggaggaaatgatgtatctgatctacactcatctgaagaaacgaataccaaaatgaaaaataatgaagaaatgatgatcggcgaggcaatggctgaaactggccatgatggtgaaacagagaatgagggcataactaccaaaacctcaaagcctgatgaagctgaaacaaacatgttgactgcagaaatggacaactttgtttgtgacacagttgaaatgagcactgaagaaggaatcattgacgctaataaaactgaaacaaatactgagtatagtaaatctgaagaaaaattagataacaatcaaatggtaatggaaagtgatattttacaggaagatcaccatacttcacagaaagtggaggaaccatcacagtgtctggcatctggaacagctatctctgagctaataatagaagacaataatgcatctcctcaaaaactaagggaacttgatccttcacttgtgtcagcaaatgacagtcctagtggcatgcagacacgctgtgtctggtctcctttggcttctccgtctacgagcattttaaagagaggactaaaaagatcccaagaagatgaaatctcatcacctgttaataaggttcgccgtgtctcctttgcagatccaatataccaagcaggattggcagatgacattgatagacggtgctctattgttaggtcccattcttccaatagttctcccataggaaaaagtgttaaaacttctcctactacacaatctaagtcagttgacctctgtgtaacagcttctgaaatttattctaagatttcagaaatggccaaagaatccataccatgcccaacagaaagtgtttacccaccattggtgaactgtgtggcaccagttgacatcattttacctcagattacatcaaacatgtgggcaagaggcctgggacaactcattagagctaagaatataaaaactattggtgatttgagtactcttacagcatctgaaataaaaactcttcctatccgttctccaaaagtgtccaatgtaaaaaaggctctcagaatatatcatgagcagcaggtgaagactcgtggactagaagagattccagtttttgatatttctgaaaaaacagtaaatggaatagaaaataaatctttgtcacctgatgaagaaagacttgtctcagatataattgatcctgttgctttagaaattccattatccaaaaaccttctggcacagattagtgctcttgctcttcagctggattcagaagatcttcataattattcaggaagccaactatttgaaatgcacgagaaactaagttcagagggaatatttttggccacagtgtgccaatttataattgtagataaagctgaatttactgggcagcaagcttatatgctgatagaagttgatgcac

SEQ ID NO: 16 - DNA, 4023 bp, Homo sapiens
gcgtgggtcagctgatcgcgcactgagggtgcgatcccgggctccccattccttcctggggcgcctccccggcccagggccaactgggtcccggtgtcggcaggcctggggtcggcgacggctgctcttttcgttctgtcgcctgcccgatggacgagcctcccgggaagcccctcagctgtgaggagaaggaaaagttaaaggagaaattagcattcttgaaaagggaatacagcaagacactagcccgccttcagcgtgcccaaagagctgaaaagattaagcattctattaagaaaacagtagaagaacaagattgtttgtctcagcaggatctctcaccgcagctaaaacactcagaacctaaaaataaaatatgtgtttatgacaagttacacatcaaaacccatcttgatgaagaaactggagaaaagacatctatcacacttgatgttgggcctgagtcctttaaccctggagatggcccaggaggattacctatacaaagaacagatgacacccaagaacattttccccacagggtcagtgaccctagtggtgagcaaaagcagaagctgccaagcagaagaaagaagcagcagaagaggacatttatttcacaggagagagactgtgtctttggcactgattcactcagattgtctgggaaaagactaaaggaacaggaagaaatcagtagcaaaaatcctgctagatcaccagtaactgaaataagaactcaccttttaagtcttaaatctgaacttccagattctccagaaccagttacagaaattaatgaagacagtgtattaattccaccaactgcccaaccagaaaaaggtgttgatacattcctaagaagacctaatttcaccagggcgactacagttcctttacagactctatcagatagcggtagtagtcagcaccttgaacacattcctcctaaaggtagcagtgaacttactactcacgacctaaaaaacattagatttacttcacctgtaagtttggaggcacaaggcaaaaaaatgactgtctctacagataacctccttgtaaataaagctataagtaaaagtggccaactgcccacaagttctaatttagaggcaaatatttcatgttctctaaatgaactcacctacaataacttaccagcaaatgaaaaccaaaacttaaaagaacaaaatcaaacagagaaatctttaaaatctcccagtgacactcttgatggcaggaatgaaaatcttcaggaaagtgagattctaagtcaacctaagagtcttagcctggaagcaacctctcctctttctgcagaaaaacattcttgcacagtgcctgaaggccttctgtttcctgcagaatattatgttagaacaacacgaagcatgtccaattgccagaggaaagtagccgtggaggctgtcattcagagtcatttggatgtcaagaaaaaagggtttaaaaataaaaataaggatgcaagtaaaaatttaaacctttccaatgaggaaactgaccaaagtgaaattaggatgtctggcacatgcacaggacaaccaagttcaagaacctctcagaaacttctctcattaactaaagtcagctctcccgctgggcccactgaagataatgacttgtctaggaaggcagttgcccaagcacctggtagaagatacacaggaaaaagaaaatcagcctgcaccccagcatcagatcattgtgaaccacttttgccaacttctagcctgtcgattgttaacaggtccaaggaagaagtcacctcacacaaatatcagcacgaaaaattatttattcaagtgaaagggaagaaaagtcgtcatcaaaaagaggattccctttcttggagtaatagtgcttatttatccttggatgatgatgctttcacggctccatttcatagggatggaatgctgagtttaaagcaactactgtcttttctcagtatcacagactttcagttacctgatgaagactttggacctcttaagcttgaaaaagtgaagtcctgctcagaaaaaccagtggagccctttgagtcaaaaatgtttggagagagacatcttaaagagggaagctgtatttttccagaggaactgagtcctaaacgcatggatacagaaatggaggacttagaagaggaccttattgttctaccaggaaaatcacatcccaaaaggccaaactcgcaaagccagcatacaaagacgggcctttcttcatccatattactttatactcctttaaatacggttgcgcctgatgataatgacaggcctaccacagacatgtgttcacctgctttccccatcttaggtactactccagcctttggccctcaaggctcctatgaaaaagcatctacagaagttgctggacgaacttgctgcacaccccaacttgctcatttgaaagactcagtctgtcttgccagtgatactaaacaattcgacagttcaggcagcccagcaaaaccacataccaccctgcaagtgtcaggcaggcaaggacaacctacctgtgactgtgactctgtcccgccaggaacacctccacccattgagtcattcacttttaaagaaaatcagctctgtagaaacacatgccaggagctgcataaacattccgtcgaacagactgaaacagcagagcttcctgcttctgatagcataaacccaggcaacctacaattggtttcagagttaaagaatccttcaggttcctgttccgtagatgtgagtgccatgttttgggaaagagccggttgtaaagagccatgtatcataactgcttgcgaagatgtagtttctctttggaaagctctggatgcttggcagtgggaaaaactttatacctggcacttcgcagaggttccagtattacagatagttccagtgcctgatgtgtataatctcgtgtgtgtagctttgggaaatttggaaatcagagagatcagggcattgttttgttcctctgatgatgaaagtgaaaagcaagtactactgaagtctggaaatataaaagctgtgcttggcctgacaaagaggaggctagttagtagcagtgggaccctttctgatcaacaagtagaagtcatgacgtttgcagaagatggaggaggcaaagaaaaccaatttttgatgccccctgaggagactatactaacttttgctgaggtccaagggatgcaagaagctctgcttggtactactattatgaacaacattgttatttggaatttaaaaactggtcaactcctgaaaaagatgcacattgatgattcttaccaagcttcagtctgtcacaaagcctattctgaaatggggcttctctttattgtcctgagtcatccctgtgccaaagagagtgagtcgttgcgaagccctgtgtttcagctcattgtgattaaccctaagacgactctcagcgtgggtgtgatgctgtactgtcttcctccagggcaggctggcaggttcctggaaggtgacgtgaaagatcactgtgcagcagcaatcttgacttctggaacaattgccatttgggacttacttctcggtcagtgtactgccctcctcccacctgtctctgaccaacattggtcttttgtgaaatggtcgggtacagactctcatttgctggctggacaaaaagatggaaatatatttgtataccactattcataagttagggtaaagtgaaaacacaattttctggatatattgggcctcttagtattttttggagttttaaatataaaggagaatatctgaatgacacttaaaatgattgcttgtttatgtccagacagacttattttttattctaatgatggtagcaccactgatcttggatgtacatttatgtatactttgagaaaaagggttttaggttgatttttgtaatttcccacatttgtacatgtgcttttaaaggtgtacataaagcttcaaatggcaataaatatttatttttatacattcaaaaaaaaaaaaaaaa

SEQ ID NO: 17 - DNA, 3911 bp, Homo sapiens
caggtgattgactggccagctgcctgaaggagcgccaggtcctccttgctggcaggtggcgaagcccattggggcggcggtgcagaccgcggcggcggctgcggcggtctggctcgggaggcgttcctggggccaaggccatggccccgcggctgcagctggagaaggcggcctggcgctgggcggagacggtgcggcccgaggaggtgtcgcaggagcacatcgagaccgcttaccgcatctggctggagccctgcattcgcggcgtgtgcagacgaaactgcaaaggaaatccgaattgcttggttggtattggtgagcatatttggttaggagaaatagatgaaaatagttttcataacatcgatgatcccaactgtgagaggagaaaaaagaactcatttgtgggcctgactaaccttggagccacttgttatgtcaacacatttcttcaagtgtggtttctcaacttggagcttcggcaggcactctacttatgtccaagcacttgtagtgactacatgctgggagacggcatccaagaagaaaaagattatgagcctcaaacaatttgtgagcatctccagtacttgtttgccttgttgcaaaacagtaataggcgatacattgatccatcaggatttgttaaagccttgggcctggacactggacaacagcaggatgctcaagaattttcaaagctctttatgtctctattggaagatactttgtctaaacaaaagaatccagatgtgcgcaatattgttcaacagcagttctgtggagaatatgcctatgtaactgtttgcaaccagtgtggcagagagtctaagcttttgtcaaaattttatgagctggagttaaatatccaaggccacaaacagttaacagattgtatctcggaatttttgaaggaagaaaaattagaaggagacaatcgctatttttgcgagaactgtcaaagcaaacagaatgcaacaagaaagattcgacttcttagccttccttgcactctgaacttgcagctaatgcgttttgtctttgacaggcaaactggacataagaaaaagctgaatacctacattggcttctcagaaattttggatatggagccttatgtggaacataaaggtgggtcctacgtgtatgaactcagcgcagtcctcatacacagaggagtgagtgcttattctggccactacatcgcccacgtgaaagatccacagtctggtgaatggtataagtttaatgatgaagacatagaaaagatggaggggaagaaattacaactagggattgaggaagatctagcagaaccttctaagtctcagacacgtaaacccaagtgtggcaaaggaactcattgctctcgaaatgcatatatgttggtttatagactgcaaactcaagaaaagcccaacactactgttcaagttccagcctttcttcaagagctggtagatcgggataattccaaatttgaggagtggtgtattgaaatggctgagatgcgtaagcaaagtgtggataaaggaaaagcaaaacacgaagaggttaaggagctgtaccaaaggttacctgctggagctgagccctatgagtttgtctctctggaatggctgcaaaagtggttggatgaatcaacacctaccaaacctattgataatcacgcttgcctgtgttcccatgacaagcttcacccggataaaatatcaattatgaagaggatatctgaatatgcagctgacattttctatagtagatatggaggaggtccaagactaactgtgaaagccctgtgtaaggaatgtgtagtagaacgttgtcgcatattgcgtctgaagaaccaactaaatgaagattataaaactgttaataatctgctgaaagcagcagtaaagggcgatggattttgggtggggaagtcctccttgcggagttggcgccagctagctcttgaacagctggatgagcaagatggtgatgcagaacaaagcaacggaaagatgaacggtagcaccttaaataaagatgaatcaaaggaagaaagaaaagaagaggaggaattaaattttaatgaagatattctgtgtccacatggtgagttatgcatatctgaaaatgaaagaaggcttgtttctaaagaggcttggagcaaactgcagcagtactttccaaaggctcctgagtttccaagttacaaagagtgctgttcacagtgcaagattttagaaagagaaggggaagaaaatgaagccttacataagatgattgcaaacgagcaaaagacttctctcccaaatttgttccaggataaaaacagaccgtgtctcagtaactggccagaggatacggatgtcctctacatcgtgtctcagttctttgtagaagagtggcggaaatttgttagaaagcctacaagatgcagccctgtgtcatcagttgggaacagtgctcttttgtgtccccacgggggcctcatgtttacatttgcttccatgaccaaagaagattctaaacttatagctctcatatggcccagtgagtggcaaatgatacaaaagctctttgttgtggatcatgtaattaaaatcacgagaattgaagtgggagatgtaaacccttcagaaacacagtatatttctgagcccaaactctgtccagaatgcagagaaggcttattgtgtcagcagcagagggacctgcgtgaatacactcaagccaccatctatgtccataaagttgtggataataaaaaggtgatgaaggattcggctccggaactgaatgtgagtagttctgaaacagaggaggacaaggaagaagctaaaccagatggagaaaaagatccagattttaatcaaatcatgcatgcattttcagttgctccttttgaccagaatttgtcaattgatggaaagattttaagtgatgactgtgccaccctaggcacccttggcgtcattcctgaatctgtcattttattgaaggctgatgaaccaattgcagattatgctgcaatggatgatgtcatgcaagtttgtatgccagaagaagggtttaaaggtactggtcttcttggacattaatctttgaatacttgctgactgctaagaaatgaccagaggggaagaggagtttgacatgttagggcattaaagcaaaggtggatttaagaattaaaccattacatgccccttccaaaaggcagaaatccattcaaacgtgactgtcccaaatgccttatgtcaaataaagcagattgcactgatggacatcagacttgaaggaaatgtttccaattttatatttaaggggggtggtgggtgggagggggcaagtaaagacggaacaagtttagtagcagtaatagtaaatcatgtttacatatgagatttatagtcgtgggaggggaataaagttctgttatatttccttgctcgagtttcataccagatgcgttggtccataaaggattgtatcaagtagatgggacaacattctgctctgaacgaaaagtaattttagagacataacctgcttaccaatgcctgtctttgattcatattctactttcaataaagcatgaaagtgaagaacttgtcctaagtgtggaaaagtgtcttcagatttagactcttctccatgtcagctgcagcgccacccgccttacacctgcccggccgtctgtctcttggtattgggtaaaggagggggcacctgcatgtctcctgcaatgagcaaggaattatgtctcatgttttgacttcagaggctttttgctttggtgcatttcagaaaggatggagaacatttattatgtgtgaaagcatcctcttccggttttgctgttattcaaaagtgggaaatgtacctggcacgtttgaaaataaaaaatctgactacctatc

SEQ ID NO: 18 - DNA, 3682 bp, Homo sapiens
aaaactgcgactgcgcggcgtgagctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggacccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaggaaaatgaaactaggaaaggtttcttaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaaca

SEQ ID NO: 19 - DNA, 2516 bp, Homo sapiens
cggacgcgcgcgcccctccccctccccccgcgctcccaacgtgtggcggctcgcgacccccggcaacccggagaaggtctacagagcggcctgcgccacgaacaaaaggatgccacggagaaagaaaaaagttaaagaagtctccgaatctcggaacctggagaagaaggatgtggaaactaccagttctgtcagtgtgaagaggaagcgtagacttgaggatgcattcattgtgatatccgatagtgatggagaggaaccaaaggaggaaaatgggttgcagaaaacgaagacaaaacagtcgaatagagcaaagtgtttggccaaaagaaaaatcgcacagatgacagaagaagaacagtttgctctggctctcaaaatgagtgagcaggaagctagggaggtgaacagccaggaggaggaagaagaggagctcttgaggaaagccattgctgaaagcctgaatagttgccggccttctgatgcttccgctaccagatctcgacctctggccactggaccgtcttcccagtcccatcaagagaaaaccacagactctgggctcactgaaggcatatggcagctggtacctccatcactgtttaaaggctcacatatcagtcagggaaacgaggctgaggaaagagaggagccttgggaccacactgaaaaaactgaagaggagccggtctctggcagctcaggaagctgggaccagtcaagccagccagtgtttgagaatgtgaacgttaaatcttttgacagatgtactggccactcggctgagcacacacagtgtgggaagccacaggaaagtactgggaggggttctgcctttctcaaagctgtccagggtagcggggacacatctaggcactgtctacctaccctagcagatgccaaaggtctccaggacactgggggcactgtgaactatttctggggtattccattctgccctgatggagtagaccctaaccagtataccaaggtcattctctgccagttggaggtttatcaaaagagcctgaaaatggctcagaggcagctccttaataaaaaaggttttggggaaccagtgttacctagacctccttctctgatccagaatgaatgtggccaaggagagcaggctagtgagaaaaatggatgcatctcagaagatatgggagatgaagacaaagaggagaggcaggagtctagggcatctgactggcactcaaaaaccaaggatttccaggaaagctcaattaaaagcttgaaagagaaacttttgttggaggaagaaccaacaaccagtcatggtcagtcttcccaagggattgttgaagaaacttctgaagagggaaactctgtacctgcttcacaaagtgttgctgctttgaccagtaagagaagcttagtccttatgccagagagttctgcagaagaaatcactgtttgtcctgagacccagctaagttcctctgaaacttttgaccttgaaagagaagtctctccaggtagcagagatatcttggatggagtcagaataataatggcagataaggaggttggtaacaaggaagatgctgagaaggaagtagctatttctaccttctcatccagtaaccaggtatcctgcccgctatgtgaccaatgctttccacctacaaagattggacgacatgccatgtactgcaatggtctgatggaggaagatacagtattgactcggagacaaaaagaggccaagaccaagagtgacagtgggacagctgcccagacttctctagacattgacaagaatgagaagtgttacctctgtaaatccctggtcccatttagagagtatcagtgtcatgtggactcctgtctccagcttgcaaaggctgaccaaggagatggacctgaagggagtggaagagcatgttcaactgtggaggggaagtggcagcagaggctgaagaacccaaaggaaaaaggccacagtgaaggccgactccttagtttcttggaacagtctgagcacaagacttcagatgcagacatcaagtcttcagaaacaggagccttcagggtgccttcaccagggatggaagaggcaggctgcagcagagagatgcagagttctttcacacgtcgtgacttaaatgaatctcccgtcaagtcttttgtttccatttcagaagccacagattgcttagtggactttaaaaagcaagttactgtccagccaggtagtcggacacggaccaaagctggcagaggaagaaggagaaaattctgaatttctagggtccaaaagttgacaaaaccattagtaggaggggtgggccatgttctataagccatagtggtccctagttcattgttgagcaagtttttagccctgcagttttcaccaccagcaccaccagcacctacccagcattctggtttttatgttttttatgatctatgcagacaactgtgtattctgttttataacagtttgtttgaatttacttacagttaaaaaatttaaatataaaaaa

SEQ ID NO: 20 - DNA, 1865 bp, Homo sapiens
gagtgcgcacgcatcctgcccgcggcgcgcgcgcaggtcggtgcgtctgtcgggggcgcgctcgggtacctgtaccccacgtagtcgccggttaccgatcggactaagttccagtggtgatttacaagtcaagttaaaatgtccccagaagtggccttgaaccgaatatctccaatgctctcccctttcatatctagcgtggtccggaatggaaaagtgggactggatgctacaaactgtttgaggataactgacttaaaatctggctgcacatcattgactcctgggcccaactgtgaccgatttaaactgcacataccatatgctggagagacattaaagtgggatatcattttcaatgcccaatacccagaactgcctcccgattttatctttggagaagatgctgaattcctgccagacccctcagctttgcagaatcttgcctcctggaatccttcaaatcctgaatgtctcttacttgtggtgaaggaacttgtgcaacaatatcaccaattccaatgtagccgcctccgggagagctcccgcctcatgtttgaataccagacattactggaggagccacagtatggagagaacatggaaatttatgctgggaaaaaaaacaactggactggtgaattttcagctcgtttccttttgaagctgcccgtagatttcagcaatatccccacataccttctcaaggatgtaaatgaagaccctggagaagatgtggccctcctctctgttagttttgaggacactgaagccacccaggtgtaccccaagctgtacttgtcacctcgaattgagcatgcacttggaggctcctcagctcttcatatcccagcttttccaggaggaggatgtctcattgattacgttcctcaagtatgccacctgctcaccaacaaggtgcagtacgtgattcaagggtatcacaaaagaagagagtatattgctgcttttctcagtcactttggcacaggtgtcgtggaatatgatgcagaaggctttacaaaactcactctgctgctgatgtggaaagatttttgttttcttgtacacattgacctgcctctgtttttccctcgagaccagccaactctcacatttcagtccgtttatcactttaccaacagtggacagctttactcccaggcccaaaaaaattatccgtacagccccagatgggatggaaatgaaatggccaaaagagcaaagggatgccaggggagcagagatgcctgcagcccgtgggagcaagtcctggcctttgcagttgcaaaaactggctgcaagctgctccagccccagaggaactggccaagctccagagggcctccttggagggcctcagagggagagagaactgctcagtaattttgatcactttgggcttatttcaaaacctttgtccctcagttccaggaggcagcatttgccaatggaaagctctaggaaacaccagtcttgagaggtggccagccagactgcctgtccacatgcgtgtcagcacatacagccgcttcctggaagccgcctggaatgtcttcacggcagcgttttgctcacacagcagcttttgcacgccccaggcagccccgactgctgaaatccaacttgagctggctggtggtccctggatcctagagcccttcacttcgggttactccctctttcttgcctctatttcttagttggaagaaataaactcacaaattatggtgcagtaattttccggggaaagtaaagcctcaggaatgcccacgcctttcttccaaagcctttgtctctgagacctcttaagttctaagattaaatgcccctcgctgtttctcctctgaaaaaaaaaaaaaaaa

SEQ ID NO: 21 - DNA, 2407 bp, Homo sapiens
gccaagatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaataaatcaggagacaaaatggggaaagatgaaaatatccagtgtaaagttacttaagctaaatcaatttcaaagaagaaaaacttggaggactcattttacctgacttcaagacttactataaagctatagtaatcaagatagatggtattggcagaggaacagacacatacgtcaatggaacagatgagagaacccagaaataaacccatataaatatgctcagctgattttgaaaaagtgaaaaagcaattcaatggaggaagaatagcctttctgacaaattatgctagagcaattagacacccatggcgaggagaaaaaagaacctctacttaaacctcacatcttatataaaatttaactcaaaatgtataacggacttaaatgtgatacataaaactagataactttgaaaaaagccacaggagaaaaatcttcaggatcttgggctaggtgacaagttcttggactttgccccgaaagcacatccataaaagacaaaatctgatatattggacttcttcaaaatttaaaaacttgtgatttaagaagaggaaaagataagctacagattgagatgaatttgcaaaccatatatctgatcaatttggaatatataaagtgtactaaaaactcaactgaagtcaggcatggtagctcatgcttgtaatctcaccactttgggaggccaagatgggaggagtgcttgaggttaggcgttccagaccagcctgggcaacatagtgagactcttgtctctacaaaaagtttttttaaaaaattaactgggcaccatgacacacaccagtagtcccagctactagggaggcaggaggatcacttgagcccaggagtttgaggcctgcggtgagctgtgatcacaccaccacactccaacctgtgtaacagagtgaggcctcatctcaaaaaaaaaaaaaaggccacaaaactcaacaataaaaacaaacagtccaattagaaaatgggcaaaagacatgaatagatgtttcactgaagaggatctatagatggcaagtaagcatatgaaaagctgttaaactccataagtcatcagggaatgcaaattgaaaccacagcgaggctatgacttacttatctcaatggctaaagaaaaaatagtgaaaataccaaatactgatgaggatacaaactggatattttatacattgctgacaggaatgtaaaatggtacagccactctgggaaagagtttatgaatttcttatcaagttaaacataattttttaatcaagttaaacataagacccagcagttgtgctcctggacattcattccagagaaatgaaaacctatattgtacttgtactcaaatattcataggagctttatttgtaatagccccaaactggaaacaacccagatgtcctacaacaggtacatggttaaacaaaccatccataacttggaatactgctctggaatgaaaaggaactaactgttgatacaagaacttggttgtacctcaggggtattatggtgaacaaa

SEQ ID NO: 22 - DNA, 2530 bp, Homo sapiens
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 23 - RNA, 21 nt, Artificial Sequence
  siRNA oligonucleotide
aacuuaggugaagcagcaucu

SEQ ID NO: 24 - RNA, 21 nt, Artificial Sequence
  siRNA oligonucleotide
aagaggaaggaccgaguagaa

SEQ ID NO: 25 - RNA, 21 nt, Artificial Sequence
  siRNA oligonucleotide
aaggugcaguacgugauucaa

SEQ ID NO: 26 - RNA, 21 nt, Artificial Sequence
  siRNA oligonucleotide
aaguuacucagccaagaacga